(19) United States
(12) Patent Application Publication
(10) Pub. No.: US 2008/0206253 A1
(43) Pub. Date: Aug. 28, 2008

(54) Method For Preventing or Treating Pain in a Mammal

(76) Inventors: Xiao-Ying Hua, La Jolla, CA (US); Camilla Svensson, San Diego, CA (US); Tony Lee Yaksh, San Diego, CA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/908,436

(22) PCT Filed: Mar. 14, 2006
(86) PCT No.: PCT/US06/09340

§ 371 Date: Sep. 12, 2007

Related U.S. Application Data
(60) Provisional application No. 60/662,105, filed on Mar. 14, 2005.

(51) Int. Cl. A61K 39/395; A61K 31/7052; A61K 38/02; A61P 25/00; C12Q 1/48; A61K 38/47

(52) U.S. Cl. 424/141.1; 514/44; 424/94.61; 424/130.1; 514/2; 435/15

(57) ABSTRACT

Methods are provided for preventing or treating pain in a mammal comprising administering an inhibitor of p38β MAP kinase in a therapeutically effective amount to the mammal in need thereof.

### CROSS REFERENCE TO RELATED APPLICATION

This application claims the benefit of the filing date of U.S. Provisional Application No. 60/662,105, filed Mar. 14, 2005, which is hereby incorporated by reference in its entirety.

### STATEMENT OF GOVERNMENT SUPPORT

This invention was made with Government support by Grant No. NS16541, awarded by the National Institutes of Health. The Government has certain rights in this invention.

### FIELD

This invention generally relates to methods for preventing or treating pain in a mammal comprising administering an inhibitor of p38β MAP kinase in a therapeutically effective amount to the mammal in need thereof.

### BACKGROUND

p38 is a member of the mitogen-activated protein kinase (MAPK) family which is known to regulate events associated with cellular stress. Shi and Gaestel, Biol Chem 383: 1519-1536, 2002. To date, four different p38 isoforms, α, β, γ and δ, have been. Hale et al., J Immunol 162: 4246-4252, 1999; Kumar et al., Nat Rev Drug Discov 2: 717-726, 2003. These isoforms have been found in peripheral tissues and, in particular p38α, associated with local inflammatory cascades. Kumar et al., Nat Rev Drug Discov 2: 717-726, 2003. The isoforms differ in their substrate preference, activation modes, and response to inhibitors. Goedert et al., EMBO J. 16: 3563-3571, 1997; Kuma et al., Biochem J 379: 133-139, 2004; Pramanik et al., J Biol Chem 278: 4831-4839, 2003; Pratt et al., J Biol Chem 278: 51928-51936, 2003; Vachon et al., Gastroenterology 123: 1980-1991, 2002. In the mature central nervous system, only p38α and p38β are constitutively expressed and it has been shown in mouse brain, that both p38α and p38β are present in neurons, while p38β is also highly expressed in glial cells. Carboni et al., Brain Res Mol Brain Res 60: 57-68, 1998; Hu et al., J Biol Chem 274: 7095-7102, 1999; Jiang et al., J Biol Chem 271: 17920-17926, 1996; Jiang et al., J Biol Chem 272: 30122-30128, 1997; Lee et al., J Neurosci Res 60: 623-631, 2000a.; Lee et al., Cytokine 12: 874-880, 2000b.

Accumulating evidence now suggest that p38 plays an important role in nociceptive processing. Pain states arising from tissue injury and inflammation are characterized by an enhanced response to subsequent afferent stimulation. This hyperalgesia arises in large part from a facilitated processing of noxious input at the spinal level. Thus injury-evoked afferent input leads to spinal release of peptides (i.e. substance P, SP) and excitatory amino acids (i.e. glutamate) that activate, through their respective receptors, signaling pathways that generate spinal sensitization. In this fashion, products of the p38-phospholipase A₂ (PLA₂)-cyclooxygenase-2 (COX-2) cascade has been shown facilitate dorsal horn activity. Svensson et al., Neuroreport 14: 1153-1157, 2003; Svensson and Yaksh, Annu Rev Pharmacol Toxicol 42: 553-583, 2002. An important element of the induction and maintenance of hyperalgesia appears to involve non-neuronal cells such as microglia and astrocytes. Watkins et al., Pain 93: 201-205, 2001. Several lines of evidence suggest that activation of spinal p38 is an important component in this process. Thus, afferent input generated by tissue and nerve injury or the direct activation of spinal neurokinin-1 (NK-1) receptor or N-methyl-D-aspartate (NMDA) receptors leads to phosphorylation (activation) of p38 in spinal microglia. Jin et al., J Neurosci 23: 4017-4022, 2003; Kim et al., Neuroreport 13: 2483-2486, 2002; Schafers et al., J Neurosci 23: 2517-2521, 2003; Tsuda et al., Glia 45: 89-95, 2004; Svensson et al., J Neurochem 86: 1534-1544, 2003b.; Svensson et al., Neuroreport 14: 1153-1157, 2003a. The hyperalgesia observed in these various behavioral models was prevented by spinal administration of p38 inhibitors, indicating a behavioral consequence of spinal p38 activation. An important issue that has not been addressed as of yet is which, if not both, of the p38 isoforms that are responsible for the observed actions.

Structurally the MAPKs (ERK, JNK and p38) display significant homology, though the ATP binding site necessary for MAPK function is sufficiently distinct as to permit the synthesis of inhibitors selective for p38. Fitzgerald et al., Nat Struct Biol 10: 764-769, 2003. However, for the p38α and p38β isoforms the structural similarities are such as to make it difficult to target either one. Thus, the majority of currently available inhibitors are mixed or equally active against the two isoforms. Even those reported to be isoform selective do not exceed IC₅₀ ratios of 20-30. Ju et al., J Pharmacol Exp Ther 301: 15-20, 2002; PCT International Application WO 2004/021988. In addition, viable p38 isoform deficient mice are not available. Adams et al., Mol Cell 6: 109-116, 2000; Mudgett et al., Proc Natl Acad Sci U S A 97: 10454-10459, 2000; Svensson et al., Abstract to the Society of Neuroscience, October 2004. A need exists in the art for specific MAP kinase inhibitors and for isoform-specific MAP kinase inhibitors that can provide a therapeutically effective treatment for pain or post-injury hyperalgesia and can lead to a reduction or prevention of pain in a mammalian subject.

### SUMMARY

The present invention provides methods for preventing or treating pain in a mammal comprising administering an inhibitor of p38β mitogen-activated protein (MAP) kinase in a therapeutically effective amount to the mammal in need thereof. The methods for treatment of pain of the present invention are based on antagonist studies which show that spinal p38 mitogen-activated protein (MAP) kinase isoforms plays a role in spinal sensitization. The present invention demonstrates that the isoforms are distinctly expressed in spinal dorsal horn: p38α in neurons and p38β in microglia. In lieu of isoform selective inhibitors, the functional role of these two individual isoforms in nociception was examined by using intrathecal isoform-specific antisense oligonucleiotides to selectively block the respective spinal isoform expression. In these rats, down-regulation of p38β, but not p38α, prevented nocifensive flinching evoked by paw formalin, hyperalgesia induced by activation of spinal neurokinin-1 receptors through intrathecal injection of substance P(SP) and the associated increases in spinal p38 phosphorylation. Thus, spinal p38β, likely in microglia, plays a significant role in spinal nociceptive processing, and represents a therapeutic target for treatment of pain.

A method for preventing or treating pain in a mammal is provided comprising administering an inhibitor of p38β MAP kinase in a therapeutically effective amount to the mammal in need thereof. In one aspect, the inhibitor is interfering RNA, short hairpin RNA, ribozyme, antisense oligonucleotide, or protein inhibitor. In a further aspect, the inhibitor is a monoclonal antibody, a polyclonal antibody, a peptide, or a small molecule. In a detailed aspect, the inhibitor exhibits an IC₅₀ value for p38β kinase that is at least ten fold less than the IC₅₀ value the inhibitor exhibits relative to other isoforms of p38 MAP kinase.

In a further embodiment the inhibitor is a compound that decreases the enzymatic activity or phosphorylation level of a p38β MAP kinase in the central nervous system of the mammal. In one aspect, the pain is hyperalgesia, allodynia or a nociceptive event.

In a further embodiment the method comprises administering an inhibitor of p38α MAP kinase.

The method further provides administering the compound intrathecally, intramedullarly, intracerebrally, intracerebroventricularly, intracranially, epidurally, intraspinally, or intraparietally. In one aspect, the compound crosses the blood-brain barrier of the mammal. In a further aspect, the compound is administered systemically. In a detailed aspect, the contacting step comprises administering the compound intravenously, parenterally, subcutaneously, intramuscularly, ophthalmicly, intraventricularly, intraperitoneally, orally, topically, or intranasally to the mammal.

In a further aspect, the p38β MAP kinase inhibitor is administered in an encapsulated form in a lipophilic compound or liposome. In a further aspect, the MAP kinase inhibitor is encapsulated in a polymer.

A method for preventing a facilitative state for sensation of pain in a mammal comprising administering an inhibitor of p38β MAP kinase in a therapeutically effective amount to the mammal in need thereof. In one aspect, the inhibitor is interfering RNA, short hairpin RNA, ribozyme, antisense oligonucleotide, or protein inhibitor. In a further aspect, the inhibitor is a monoclonal antibody, a polyclonal antibody, a peptide, or a small molecule. In a detailed aspect, the inhibitor exhibits an IC₅₀ value for p38β kinase that is at least ten fold less than the IC₅₀ value the inhibitor exhibits relative to other isoforms of p38 MAP kinase. In a further aspect, the method comprises administering an inhibitor of p38β MAP kinase.

A method of treating, reducing, or preventing pain in a mammalian subject is provided comprising contacting the periphery of the mammalian subject with a compound that decreases the enzymatic activity or phosphorylation level of a p38β MAP kinase in the central nervous system of the mammal, in an amount sufficient to treat, reduce, or prevent pain.

The method further provides administering the compound intrathecally, intramedullarly, intracerebrally, intracerebroventricularly, intracranially, epidurally, intraspinally, or intraparietally. In one aspect, the compound crosses the blood-brain barrier of the mammal. In a further aspect, the compound is administered systemically. In a detailed aspect, the contacting step comprises administering the compound intravenously, parenterally, subcutaneously, intramuscularly, ophthalmicly, intraventricularly, intraperitoneally, orally, topically, or intranasally to the mammal. In a further aspect, the method comprises administering an inhibitor of p38α MAP kinase.

A method for identifying a compound which inhibits p38β MAP kinase is provided comprising contacting a test compound with a cell-based assay system comprising a cell expressing p38β MAP kinase and capable of signaling responsiveness to p38β MAP kinase, and detecting an effect of the test compound on p38β MAP kinase signaling in the assay system, effectiveness of the test compound in the assay being indicative of the inhibition. In one aspect, the test compound is a monoclonal antibody, a polyclonal antibody, a peptide, a nucleic acid, or a small molecule.

In one embodiment, a pharmaceutical composition comprising a p38β MAP kinase inhibitor for treatment of pain. In one aspect, the pain is hyperalgesia. In a further aspect, the pain is allodynia. In a one aspect, the inhibitor is interfering RNA, short hairpin RNA, ribozyme, antisense oligonucleotide, or protein inhibitor. In a further aspect, the inhibitor is a monoclonal antibody, a polyclonal antibody, a peptide, or a small molecule. In a detailed aspect, the antisense oligonucleotide is 5′-GTATGTCCTCCTCGCGTGGA-3′.

### BRIEF DESCRIPTION OF THE DRAWINGS

FIG. 1 shows protein expression of p38α and p38β in naive rat spinal cord.

FIG. 2 shows distribution and cellular localization of p38α in naive rat spinal cord.

FIG. 3 shows distribution and cellular localization of p38β in naive rat spinal cord.

FIG. 4 shows effect of intrathecal (IT) delivery of p38 isoform specific antisense oligonucleotides (AS) on spinal p38α and p38β protein expression.

FIG. 5 shows effect of IT p38α and p38β antisense oligonucleotides (AS) and SB203580, a p38 inhibitor, on formalin-induced hyperalgesia.

FIG. 6 shows effect of IT p38 isoform specific antisense oligonucleotides (AS) on phosphorylation of spinal p38 evoked by injection of formalin to the hind paw.

FIG. 7 shows effect of IT p38α and p38β antisense oligonucleotides (AS) on thermal hyperalgesia and spinal p38 phosphorylation evoked by IT substance P(SP).

FIG. 8 shows inhibition of spinal p38α/β attenuates SP-evoked spinal PGE2 release.

### DETAILED DESCRIPTION

The present invention provides methods for preventing or treating pain in a mammal comprising administering an inhibitor of p38β MAP kinase in a therapeutically effective amount to the mammal in need thereof. The methods for treatment of pain of the present invention are based on antagonist studies which show that spinal p38 mitogen-activated protein (MAP) kinase isoforms plays a role in spinal sensitization. The present invention demonstrates that the isoforms are distinctly expressed in spinal dorsal horn: p38α in neurons and p38β in microglia. Spinal p38β plays a significant role in spinal nociceptive processing, and represents a therapeutic target for treatment of pain, hyperalgesia, allodynia, or nociceptive events.

In order to define the role of spinal p38α/β isoforms in nociception, a series of experiments were performed with several specific aims: (i) to examine the anatomical and cellular distribution and location of p38α/β isoforms in rat spinal cord; (ii) to verify the efficacy and specificity of intrathecal (IT) treatment with antisense oligonucleotides (AS) on spinal expression of these two isoforms; (iii) to examine the effect of down-regulation of individual isoform on nocifensive behaviors in two experimental models of hyperalgesia; (iv) to determine which isoform contribute to the stimuli-induced spinal p38 activation; and (v) to study possible mechanism of p38 mediating spinal sensitization.

It is to be understood that this invention is not limited to particular methods, reagents, compounds, compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of ±20% or ±10%, more preferably ±5%, even more preferably 1%, and still more preferably +0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Hyperalgesia” refers to increased sensitivity and lowered threshold to pain stimuli such as occurs in a superficial burn of the skin or with Complex Regional Pain Syndrome type one (CRPS I) or formerly Reflex Sympathetic Dystrophy (RSD)

“Allodynia” refers to pain due to a stimulus that does not normally provoke pain. The original definition adopted by the IASP committee was pain due to non-noxious stimulus to the normal skin. Allodynia involves a change in the quality of a sensation, tactile, thermal, or of any other kind. The usual response to a stimulus was not painful, but the present response is.

“IC₅₀” refers to an amount, concentration, or dosage of a particular test compound that achieves 50% inhibition of a maximal response in an assay that measure such a response.

“Nociceptive event” refers to painful or injurious stimuli directly or indirectly causing the transmission of pain. “Nociceptive” refers to the process of pain transmission; usually relating to a receptive neuron for painful sensations.

“Preemptive analgesia” refers to the administration of anti-pain therapy prior to the first nociceptive event and, without being bound by any theory, likely preventing or reducing the activation of the nociceptors.

“Prevention or treatment of pain” refers to inhibition and/or alleviation of pain sensation.

Any form of pain, chronic or acute, can be treated by the methods of the present invention. Pain states susceptible to treatment by the present method include, but are not limited to, neurological pain, neuropathies, polyneuropathies, diabetes-related polyneuropathies, headache (migraine and tension), trauma, neuralgias, post-zosterian neuralgia, trigeminal neuralgia, algodystrophy, HTV-related pain, musculo-skeletal pain, osteo-traumatic pain (e.g., bone fractures), arthritis, fibromyalgia, osteoarthritis, rheumatoid arthritis, spondylarthritis, phantom limb pain, back pain, vertebral pain, slipped disc surgery failure, post-surgery pain, cancer-related pain, vascular pain, Raynaud's syndrome, Horton's disease, arthritis, varicose ulcers, visceral pain, and childbirth.

Additionally, any form of anticipated pain may be prevented by the methods of the present invention. Preferably, the present method is used to prevent pain associated with surgery. Early intervention therapy is commonly known as preemptive analgesia, which reduces the hypersensitization of nociceptors by blocking pain impulses from ever reaching the brain.

Preemptive analgesia has received widespread acceptance as an adjunct to reduce perioperative pain in patients who undergo dental and surgical procedures, such as generally disclosed by Mayer et al. in U.S. Pat. No. 5,502,058. The technique is well accepted and is believed to involve the pharmacological interruption of afferent neurons to the dorsal horns of the spinal cord prior to the delivery of painful stimuli, such as a surgical incision. The anesthetic concept can be applied to most dental or surgical procedures, minimizing postoperative pain and the necessity for narcotic or parenteral analgesia, as well as reducing hospitalizations and required convalescence.

“Inhibitors,” “activators,” and “modulators” of p38β MAP kinase signaling refer to inhibitory, activating, or modulating molecules, respectively, identified using in vitro and in vivo assays for p38β MAP kinase binding or signaling, e.g., ligands, agonists, antagonists, and their homologs and mimetics.

“Modulator” includes inhibitors and activators. Inhibitors refers to agents that, e.g., bind to, partially or totally block stimulation, decrease, prevent, delay activation, inactivate, desensitize, or down regulate the activity of p38β MAP kinase signaling, e.g., antagonists. Activators are agents that, e.g., bind to, stimulate, increase, open, activate, facilitate, enhance activation, sensitize or up regulate the activity of p38β MAP kinase signaling, e.g., agonists. Modulators include agents that, e.g., alter the interaction of p38β MAP kinase with: proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring p38β MAP kinase ligands, e.g., with altered activity, as well as naturally occurring and synthetic ligands, antagonists, agonists, small chemical molecules and the like. Such assays for inhibitors and activators include, e.g., applying putative modulator compounds to a cell expressing a p38β MAP kinase and then determining the functional effects on p38β MAP kinase signaling, as described herein. Samples or assays comprising p38β MAP kinase that are treated with a potential activator, inhibitor, or modulator are compared to control samples without the inhibitor, activator, or modulator to examine the extent of inhibition. Control samples (untreated with inhibitors) can be assigned a relative p38β MAP kinase activity value of 100%. Inhibition of p38β MAP kinase is achieved when the p38β MAP kinase activity value relative to the control is about 80%, optionally 50% or 25-0%. Activation of p38β MAP kinase is achieved when the p38β MAP kinase activity value relative to the control is 110%, optionally 150%, optionally 200-500%, or 1000-3000% higher. Exemplary p38β MAP kinase binding activity assays of the present invention are: a p38β MAP kinase ligand blot assay (Aymerich et al., Invest Ophthalinol Vis Sci. 42: 3287-93, 2001); a p38β MAP kinase affinity column chromatography assay (Alberdi, J Biol Chem. 274: 31605-12, 1999) and a p38β MAP kinase ligand binding assay (Alberdi et al., J Biol Chem. 274: 31605-12, 1999). Each incorporated by reference in their entirety.

“Signaling in cells” refers to the interaction of a ligand, such as an endogenous or exogenous ligand, with p38β MAP kinase resulting in cell signaling to produce a response, for example, pain or a nociceptive response.

“Test compound” refers to a nucleic acid, DNA, RNA, protein, polypeptide, or small chemical entity that is determined to effect an increase or decrease in a gene expression as a result of signaling through the p38β MAP kinase pathway. The test compound can be an antisense RNA, ribozyme, polypeptide, or small molecular chemical entity. “Test compound” can be any small chemical compound, or a biological entity, such as a protein, sugar, nucleic acid or lipid. Typically, test compounds will be small chemical molecules and polypeptides. A “test compound specific for signaling by p38β MAP kinase is determined to be a modulator of p38β MAP kinase pathway signaling via ligand.

“Cell-based assays” include p38β MAP kinase binding assays, for example, radioligand or fluorescent ligand binding assays for p38β MAP kinase to cells, plasma membranes, detergent-solubilized plasma membrane proteins, immobilized collagen (Alberdi et al., 1999, JBC; Meyer et al., 2002); p38β MAP kinase-affinity column chromatography (Alberdi et al., 1999, JBC; Aymerich et al., 2001); p38α MAP kinase ligand blot using a radio- or fluoresceinated-ligand (Aymerich et al., 2001; Meyer et al., 2002); Size-exclusion ultrafiltration (Alberdi et al., 1998, Biochem.; Meyer et al., 2002); or ELISA. Cell-based assays further include, but are not limited to TNF cellular assay, p38β MAP kinase binding assay, fatty acid translocator assay, or thrombospondin binding assay. Exemplary in vitro assays can be found, for example, in PCT International application WO 2004/021988, all of which are incorporated herein in their entirety.

“Detecting an effect” refers to an effect measured in a cell-based assay system. For example, the effect detected can be p38β MAP kinase activity in an assay system, for example, p38β MAP kinase binding assay.

“Assay being indicative of modulation” refers to results of a cell-based assay system indicating that inhibition of cell activation by p38β MAP kinase pathway signaling induces a protective response in cells against pain or a nociceptive response.

“Biological activity” and “biologically active” with regard to a p38β MAP kinase activity of the present invention refer to the ability of a molecule to specifically bind to and signal through a native or recombinant p38β MAP kinase, or to block the ability of a native or recombinant p38β MAP kinase to participate in signal transduction. Thus, the (native and variant) p38β MAP kinase ligands of the present invention include agonists and antagonists of a native or recombinant p38β MAP kinase. Preferred biological activities of the p38β MAP kinase ligands of the present invention include the ability to inhibit, for example, pain or hyperalgesia. Accordingly, the administration of the compounds or agents of the present invention can prevent or delay, to alleviate, or to arrest or inhibit development of the symptoms or conditions associated with pain, hyperalgesia, allodynia, nociceptive events, or other disorders.

“High affinity” for a ligand refers to an equilibrium association constant (Ka) of at least about 103M-1, at least about 104M-1, at least about 105M-1, at least about 106M-1, at least about 107M-1, at least about 108M-1, at least about 109M-1, at least about 1010M-1, at least about 1011M-1, or at least about 1012M-1 or greater, e.g., up to 1013M-1 or 1014M-1 or greater. However, “high affinity” binding can vary for other ligands.

“Ka”, as used herein, is intended to refer to the equilibrium association constant of a particular ligand-receptor interaction, e.g., antibody-antigen interaction. This constant has units of 1/M.

“Kd”, as used herein, is intended to refer to the equilibrium dissociation constant of a particular ligand-receptor interaction. This constant has units of M.

“ka”, as used herein, is intended to refer to the kinetic association constant of a particular ligand-receptor interaction. This constant has units of 1/Ms.

“kd”, as used herein, is intended to refer to the kinetic dissociation constant of a particular ligand-receptor interaction. This constant has units of 1/s.

“Particular ligand-receptor interactions” refers to the experimental conditions under which the equilibrium and kinetic constants are measured.

“Isotype” refers to the antibody class that is encoded by heavy chain constant region genes. Heavy chains are classified as gamma, mu, alpha, delta, or epsilon, and define the antibody's isotype as IgG, IgM, IgA, IgD and IgE, respectively. Additional structural variations characterize distinct subtypes of IgG (e.g., IgG1, IgG2, IgG3 and IgG4) and IgA (e.g., IgA1 and IgA2)

“Antagonist” is used in the broadest sense, and includes any molecule that partially or fully blocks, inhibits, or neutralizes a biological activity of a p38, MAP kinase polypeptide. In a similar manner, the term “agonist” is used in the broadest sense and includes any molecule that mimics or enhances a biological activity of a p38β MAP kinase polypeptide. Suitable antagonist molecules specifically include antagonist antibodies or antibody fragments, fragments or amino acid sequence variants of native p38β MAP kinase polypeptides, peptides, antisense oligonucleotides, small organic molecules, and the like. Methods for identifying antagonists of a p38βMAP kinase polypeptide can comprise contacting a p38β MAP kinase polypeptide with a candidate antagonist molecule and measuring a detectable change in one or more biological activities normally associated with the p38β MAP kinase polypeptide.

The ability of a molecule to bind to p3813 MAP kinase can be determined, for example, by the ability of the putative ligand to bind to p38β MAP kinase immunoadhesin coated on an assay plate. Specificity of binding can be determined by comparing binding to p3813 MAP kinase.

“Sorting” in the context of cells as used herein to refers to both physical sorting of the cells, as can be accomplished using, e.g., a fluorescence activated cell sorter, as well as to analysis of cells based on expression of cell surface markers, e.g., FACS analysis in the absence of sorting.

“Cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny cannot be precisely identical in DNA content, due to deliberate or inadvertent mutations. Mutant progeny that have the same function or biological activity as screened for in the originally transformed cell are included. Where distinct designations are intended, it will be clear from the context.

“Treating” refers to any indicia of success in the treatment or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination. Accordingly, the term “treating” includes the administration of the compounds or agents of the present invention to treat pain, hyperalgesia, allodynia, or nociceptive events. Accordingly, the term “treating” includes the administration of the compounds or agents of the present invention to prevent or delay, to alleviate, or to arrest or inhibit development of the symptoms or conditions associated with pain, hyperalgesia, allodynia, nociceptive events, or other disorders. The term “therapeutic effect” refers to the reduction, elimination, or prevention of the disease, symptoms of the disease, or side effects of the disease in the subject.

“Periphery of a mammal” refers to any part of the body outside the central nervous system of the mammal.

“Concomitant administration” of a known drug with a compound of the present invention means administration of the drug and the compound at such time that both the known drug and the compound will have a therapeutic effect or diagnostic effect. Such concomitant administration can involve concurrent (i.e., at the same time), prior, or subsequent administration of the drug with respect to the administration of a compound of the present invention. A person of ordinary skill in the art, would have no difficulty determining the appropriate timing, sequence and dosages of administration for particular drugs and compounds of the present invention.

“Subject”, “patient” or “mammal” refer to any mammalian patient or subject to which the compositions of the invention can be administered. The term mammal refers to human patients and non-human primates, as well as experimental animals such as rabbits, rats, and mice, and other animals. In an exemplary embodiment, of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that can be associated with the targeted or suspected disease or condition. These and other routine methods allow the clinician to select patients in need of therapy using the methods and formulations of the invention.

“Specifically (or selectively) binds” to an antibody refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample.

“Specifically bind(s)” or “bind(s) specifically” when referring to a peptide or small molecule refers to a peptide molecule or small molecule which has intermediate or high binding affinity, exclusively or predominately, to a target molecule. The phrase “specifically binds to” refers to a binding reaction which is determinative of the presence of a target protein in the presence of a heterogeneous population of proteins and other biologics. Thus, under designated assay conditions, the specified binding moieties bind preferentially to a particular target protein and do not bind in a significant amount to other components present in a test sample. Specific binding to a target protein under such conditions can require a binding moiety that is selected for its specificity for a particular target antigen. A variety of assay formats can be used to select ligands that are specifically reactive with a particular protein. For example, solid-phase ELISA immunoassays, immunoprecipitation, Biacore and Western blot are used to identify peptides that specifically react with p38β MAP kinase domain-containing proteins. Typically a specific or selective reaction will be at least twice background signal or noise and more typically more than 10 times background. Specific binding between a monovalent peptide and p38β MAP kinase means a binding affinity of at least 103 M-1, and preferably 105, 106, 107, 108, 109 or 1010 M-1.

This invention relies on routine techniques in the field of recombinant genetics. Basic texts disclosing the general methods of use in this invention include Sambrook et al., Molecular Cloning, A Laboratory Manual, 2nd ed., 1989; Kriegler, Gene Transfer and Expression: A Laboratory Manual, 1990; and Ausubel et al., eds., Current Protocols in Molecular Biology, 1994.

p38β MAP kinase nucleic acids, polymorphic variants, orthologs, and alleles that are substantially identical to sequences provided herein can be isolated using p38β MAP kinase nucleic acid probes and oligonucleotides under stringent hybridization conditions, by screening libraries. Alternatively, expression libraries can be used to clone p38β MAP kinase protein, polymorphic variants, orthologs, and alleles by detecting expressed homologs immunologically with antisera or purified antibodies made against human p38β MAP kinase or portions thereof.

### Identification of Compounds for Treatment and Prophylaxis of Disease

Identifying bioactive agents that modulate p38β MAP kinase signaling, the information is used in a wide variety of ways. In one method, one of several cellular assays, e.g., p38β MAP kinase assay, can be used in conjunction with high throughput screening techniques, to allow monitoring for antagonists of p38β MAP kinase pathway signaling after treatment with a candidate agent, Zlokarnik, et al., Science 279:84-8, 1998; and Heid et al., Genome Res. 6:986, 1996; each incorporated herein by reference in their entirety. In one method, the candidate agents are added to cells.

The term “candidate bioactive agent” or “drug candidate” or grammatical equivalents as used herein describes any molecule, e.g., protein, oligopeptide, small organic molecule, polysaccharide, polynucleotide, to be tested for bioactive agents that are capable of directly or indirectly altering the activity of p38β MAP kinase signaling. In one methods, the bioactive agents modulate p38β MAP kinase signaling. In a further embodiment of the method, the candidate agents induce an antagonist effect in a p38β MAP kinase assay, as further described below. Generally a plurality of assay mixtures are run in parallel with different agent concentrations to obtain a differential response to the various concentrations. Typically, one of these concentrations serves as a negative control, i.e., at zero concentration or below the level of detection.

Candidate agents encompass numerous chemical classes, though typically they are organic molecules, e.g., small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents can be subjected to directed or random chemical modifications, such as acylation, alkylation, esterification, amidification to produce structural analogs.

In some embodiments, the candidate bioactive agents are proteins. By “protein” herein is meant at least two covalently attached amino acids, which includes proteins, polypeptides, oligopeptides and peptides. The protein can be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the methods herein. “Amino acid” also includes imino acid residues such as proline and hydroxyproline. The side chains can be in either the (R) or the (S) configuration. In further embodiments, the amino acids are in the (S) or L-configuration. If non-naturally occurring side chains are used, non-amino acid substituents can be used, for example to prevent or retard in vivo degradations.

In one method, the candidate bioactive agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening using the methods herein. The libraries can be bacterial, fungal, viral, and mammalian proteins, and human proteins.

In some methods, the candidate bioactive agents are peptides of from about 5 to about 30 amino acids, typically from about 5 to about 20 amino acids, and typically from about 7 to about 15 being. The peptides can be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they can incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In some methods, the library can be fully randomized, with no sequence preferences or constants at any position. In other methods, the library can be biased. Some positions within the sequence are either held constant, or are selected from a limited number of possibilities. For example, in some methods, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, or to purines. In other methods, the candidate bioactive agents are nucleic acids, as defined above.

As described above generally for proteins, nucleic acid candidate bioactive agents can be naturally occurring nucleic acids, random nucleic acids, or “biased” random nucleic acids. For example, digests of procaryotic or eucaryotic genomes can be used as is outlined above for proteins.

In some methods, the candidate bioactive agents are organic chemical moieties.

(A) Drug Screening Methods

Several different drug screening methods can be accomplished to identify drugs or bioactive agents that inhibit p38β MAP kinase signaling. One such method is the screening of candidate agents that can act as an antagonist p38β MAP kinase signaling, thus generating the associated phenotype. Candidate agents that can act as an antagonist to p38β MAP kinase pathway signaling, as shown herein, are expected to prevent a facilitative state for sensation of pain, e.g., hyperalgesia, allodynia, or nociceptive events. Thus, in some methods, candidate agents can be determined alter p38β MAP kinase pathway signaling.

Thus, screening of candidate agents that modulate p38β MAP kinase pathway signaling either at the level of gene expression or protein level can be accomplished.

In some methods, a candidate agent can be administered in any one of several cellular assays, e.g., p38β MAP kinase assay. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e., a peptide) can be put into a viral construct such as a retroviral construct and added to the cell, such that expression of the peptide agent is accomplished; see PCT US97/01019, incorporated herein by reference in its entirety.

Once the candidate agent has been administered to the cells, the cells can be washed if desired and are allowed to incubate under physiological conditions for some period of time. The cells are then harvested and a new gene expression profile is generated, as outlined herein.

For example, p38β MAP kinase signaling can be screened for agents that prevent a facilitative state for sensation of pain, e.g., hyperalgesia, allodynia, or nociceptive events. A change in a binding assay or cellular assay indicates that the agent has an effect on p38β MAP kinase pathway signaling activity. In one method, an anti-nociceptive profile is induced or maintained, before, during, and/or after stimulation with antigen. By defining such a signature for anti-nociceptive response, screens for new drugs that mimic the anti-nociceptive phenotype can be devised. With this approach, the drug target need not be known and need not be represented in the original expression screening platform, nor does the level of transcript for the target protein need to change. In some methods, the agent acts as an antagonist in one of several cellular or binding assays, e.g., p38β MAP kinase assay.

In some methods, screens can be done on individual genes and gene products. After having identified a cellular or binding assay as indicative of a facilitative state for sensation of pain, e.g., hyperalgesia or allodynia, screening of modulators of cellular or binding assay can be completed.

Thus, in some methods, screening for modulators of cellular or binding assay can be completed. This will be done as outlined above, but in general a few cellular or binding assay are evaluated. In some methods, screens are designed to first find candidate agents that can affect a cellular activity or binding assay, and then these agents can be used in other assays that evaluate the ability of the candidate agent to modulate p38β MAP kinase signaling.

In general, purified or isolated gene product can be used for binding assays; that is, the gene products of p38β MAP kinase are made. Using the nucleic acids of the methods and compositions herein which encode a p38β MAP kinase, a variety of expression vectors can be made. The expression vectors can be either self-replicating extrachromosomal vectors or vectors which integrate into a host genome. Generally, these expression vectors include transcriptional and translational regulatory nucleic acid operably linked to the nucleic acid encoding a p38β MAP kinase protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express p38β MAP kinase protein; for example, transcriptional and translational regulatory nucleic acid sequences from Bacillus are used to express the protein in Bacillus. Numerous types of appropriate expression vectors, and suitable regulatory sequences are known in the art for a variety of host cells.

In general, the transcriptional and translational regulatory sequences can include, but are not limited to, promoter sequences, ribosomal binding sites, transcriptional start and stop sequences, translational start and stop sequences, and enhancer or activator sequences. In one method, the regulatory sequences include a promoter and transcriptional start and stop sequences.

Promoter sequences encode either constitutive or inducible promoters. The promoters can be either naturally occurring promoters or hybrid promoters. Hybrid promoters, which combine elements of more than one promoter, are also known in the art, and are useful in the methods herein.

In addition, the expression vector can comprise additional elements. For example, the expression vector can have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and typically two homologous sequences which flank the expression construct. The integrating vector can be directed to a specific locus in the host cell by selecting the appropriate homologous sequence for inclusion in the vector. Constructs for integrating vectors are well known in the art. Methods to effect homologous recombination are described in PCT US93/03868 and PCT US98/05223, each incorporated herein by reference in their entirety.

In some methods, the expression vector contains a selectable marker gene to allow the selection of transformed host cells. Selection genes are well known in the art and will vary with the host cell used.

One expression vector system is a retroviral vector system such as is generally described in PCT/US97/01019 and PCT/US97/01048, each incorporated herein by reference in their entirety.

The p38β MAP kinase proteins or the inhibitors of p38β MAP kinase proteins of the present methods and compositions are produced by culturing a host cell transformed with an expression vector containing nucleic acid encoding p38β MAP kinase, under the appropriate conditions to induce or cause expression of the protein. The conditions appropriate for p38β MAP kinase expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In some methods, the timing of the harvest is important. For example, the baculoviral systems used in insect cell expression are lytic viruses, and thus harvest time selection can be crucial for product yield.

Appropriate host cells include yeast, bacteria, archebacteria, fungi, and insect and animal cells, including mammalian cells. Of particular interest are Drosophila melangaster cells, Saccharomyces cerevisiae and other yeasts, E. coli, Bacillus subtilis, SF9 cells, C129 cells, 293 cells, Neurospora, BHK, CHO, COS, and HeLa cells. In some methods, microglial or neuronal cells are host cells as provided herein, which for example, include non-recombinant cell lines, such as primary cell lines. In addition, purified primary microglial or neuronal cells for p38β MAP kinase assay derived from either transgenic or non-transgenic strains can also be used. The host cell can alternatively be an cell type known to be involved in the process of pain transmission.

In one method, the p38β MAP kinase proteins or the inhibitors of p38β MAP kinase proteins are expressed in mammalian cells. Mammalian expression systems can include retroviral systems. A mammalian promoter is any DNA sequence capable of binding mammalian RNA polymerase and initiating the downstream (3′) transcription of a coding sequence for p38β MAP kinase protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenlytion signals include those derived form SV40.

The methods of introducing nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In some methods, p38β MAP kinase proteins or the inhibitors of p38β MAP kinase proteins are expressed in bacterial systems which are well known in the art.

In other methods, p38β MAP kinase proteins or the inhibitors of p38β MAP kinase proteins can be produced in insect cells. Expression vectors for the transformation of insect cells, and in particular, baculovirus-based expression vectors, are well known in the art.

In some methods, p38β MAP kinase proteins or the inhibitors of p38β MAP kinase proteins are produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica.

A p38β MAP kinase protein or the inhibitor of p38β MAP kinase proteins can also be made as a fusion protein, using techniques well known in the art. For example, for the creation of monoclonal antibodies, if the desired epitope is small, the protein can be fused to a carrier protein to form an immunogen. Alternatively, p38β MAP kinase protein can be made as a fusion protein to increase expression. For example, when a protein is a shorter peptide, the nucleic acid encoding the peptide can be linked to other nucleic acid for expression purposes. Similarly, p38β MAP kinase proteins of the methods and compositions herein can be linked to protein labels, such as green fluorescent protein (GFP), red fluorescent protein (RFP), yellow fluorescent protein (YFP), and blue fluorescent protein (BFP).

In one embodiment, the proteins are recombinant. A “recombinant protein” is a protein made using recombinant techniques, i.e., through the expression of a recombinant nucleic acid as depicted above. A recombinant protein is distinguished from naturally occurring protein by at least one or more characteristics. For example, the protein can be isolated or purified away from some or all of the proteins and compounds with which it is normally associated in its wild type host, and thus can be substantially pure. For example, an isolated protein is unaccompanied by at least some of the material with which it is normally associated in its natural state, typically constituting at least about 0.5%, typically at least about 5% by weight of the total protein in a given sample. A substantially pure protein comprises at least about 75% by weight of the total protein, at least about 80%, and typically at least about 90%. The definition includes the production of p38β MAP kinase protein from one organism in a different organism or host cell. Alternatively, the protein can be made at a significantly higher concentration than is normally seen, through the use of a inducible promoter or high expression promoter, such that the protein is made at increased concentration levels. Alternatively, the protein can be in a form not normally found in nature, as in the addition of an epitope tag or amino acid substitutions, insertions and deletions, as discussed below.

In some methods, when the p38β MAP kinase protein is to be used to generate antibodies, the protein must share at least one epitope or determinant with the full length transcription product of the nucleic acids. By “epitope” or “determinant” herein is meant a portion of a protein which will bind an antibody. Thus, in most instances, antibodies made to a smaller protein should be able to bind to the full length protein. In one embodiment, the epitope is unique; that is, antibodies generated to a unique epitope show little or no cross-reactivity.

In some methods, the antibodies provided herein can be capable of reducing or eliminating the biological function of a p38β MAP kinase protein, as is described below. The addition of antibodies (either polyclonal or monoclonal) to the protein (or cells containing the protein) can reduce or eliminate the protein's activity. Generally, at least a 25% decrease in activity is observed, with typically at least about 50% and typically about a 95-100% decrease being observed.

In addition, the proteins can be variant proteins, comprising one more amino acid substitutions, insertions and deletions.

In one method, a p38β MAP kinase protein is purified or isolated after expression. Proteins can be isolated or purified in a variety of ways. Standard purification methods include electrophoretic, molecular, immunological and chromatographic techniques, including ion exchange, hydrophobic, affinity, and reverse-phase HPLC chromatography, and chromatofocusing. For example, a p38β MAP kinase protein can be purified using a standard anti-p38β MAP kinase protein antibody column. Ultrafiltration and diafiltration techniques, in conjunction with protein concentration, are also useful. For general guidance in suitable purification techniques, see Scopes, Protein Purification, Springer-Verlag, NY, 1982, incorporated herein by reference in its entirety. The degree of purification necessary will vary depending on the use of the protein. In some instances no purification will be necessary.

Once the gene product of the p38β MAP kinase gene is made, binding assays can be done. These methods comprise combining a p38β MAP kinase protein and a candidate bioactive agent, and determining the binding of the candidate agent to the p38β MAP kinase protein. Methods utilize a human p38β MAP kinase protein, although other mammalian proteins can also be used, including rodents (mice, rats, hamsters, guinea pigs), farm animals (cows, sheep, pigs, horses) and primates. These latter methods can be used for the development of animal models of human disease. In some methods, variant or derivative p38β MAP kinase proteins can be used, including deletion p38β MAP kinase proteins as outlined above.

The assays herein utilize p38β MAP kinase proteins as defined herein. In some assays, portions of proteins can be utilized. In other assays, portions having different activities can be used. In addition, the assays described herein can utilize either isolated p38β MAP kinase proteins or cells comprising the p38β MAP kinase proteins. In some methods, the protein or the candidate agent is non-diffusably bound to an insoluble support having isolated sample receiving areas (e.g., a microtiter plate or an array). The insoluble supports can be made of any composition to which the compositions can be bound, is readily separated from soluble material, and is otherwise compatible with the overall method of screening. The surface of such supports can be solid or porous and of any convenient shape. Examples of suitable insoluble supports include microtiter plates, arrays, membranes and beads. These are typically made of glass, plastic (e.g., polystyrene), polysaccharides, nylon or nitrocellulose, and teflon™. Microtiter plates and arrays are especially convenient because a large number of assays can be carried out simultaneously, using small amounts of reagents and samples. In some cases magnetic beads and the like are included. The particular manner of binding of the composition is not crucial so long as it is compatible with the reagents and overall methods described herein, maintains the activity of the composition and is nondiffusable. Methods of binding include the use of antibodies (which do not sterically block either the ligand binding site or activation sequence when the protein is bound to the support), direct binding to ionic supports, chemical crosslinking, or by the synthesis of the protein or agent on the surface. Following binding of the protein or agent, excess unbound material is removed by washing. The sample receiving areas can then be blocked through incubation with bovine serum albumin (BSA), casein or other innocuous protein or other moiety. Also included in the methods and compositions herein are screening assays wherein solid supports are not used.

In other methods, the p38β MAP kinase protein is bound to the support, and a candidate bioactive agent is added to the assay. Alternatively, the candidate agent is bound to the support and the protein is added. Novel binding agents include specific antibodies, non-natural binding agents identified in screens of chemical libraries, and peptide analogs. Of particular interest are screening assays for agents that have a low toxicity for human cells. A wide variety of assays can be used for this purpose, including labeled in vitro protein-protein binding assays, electrophoretic mobility shift assays, immunoassays for protein binding, functional assays (such as phosphorylation assays) and the like.

The determination of the binding of the candidate bioactive agent to a p38β MAP kinase protein can be done in a number of ways. In some methods, the candidate bioactive agent is labeled, and binding determined directly. For example, this can be done by attaching all or a portion of a p38β MAP kinase protein to a solid support, adding a labeled candidate agent (for example a fluorescent label), washing off excess reagent, and determining whether the label is present on the solid support. Various blocking and washing steps can be utilized.

By “labeled” herein is meant that the compound is either directly or indirectly labeled with a label which provides a detectable signal, e.g., radioisotope, fluorescers, enzyme, antibodies, particles such as magnetic particles, chemiluminescers, or specific binding molecules. Specific binding molecules include pairs, such as biotin and streptavidin, digoxin and antidigoxin. For the specific binding members, the complementary member would normally be labeled with a molecule which provides for detection, in accordance with known procedures, as outlined above. The label can directly or indirectly provide a detectable signal.

In some methods, only one of the components is labeled. For example, the proteins (or proteinaceous candidate agents) can be labeled at tyrosine positions using ¹²⁵I, or with fluorophores. Alternatively, more than one component can be labeled with different labels; using ¹²⁵I for the proteins, for example, and a fluorophor for the candidate agents.

In other methods, the binding of the candidate bioactive agent is determined through the use of competitive binding assays. In this method, the competitor is a binding moiety known to bind to the target molecule such as an antibody, peptide, binding partner, or ligand. Under certain circumstances, there can be competitive binding as between the bioactive agent and the binding moiety, with the binding moiety displacing the bioactive agent. This assay can be used to determine candidate agents which interfere with binding between proteins and the competitor.

In some methods, the candidate bioactive agent is labeled. Either the candidate bioactive agent, or the competitor, or both, is added first to the protein for a time sufficient to allow binding, if present. Incubations can be performed at any temperature which facilitates optimal activity, typically between about 4° C. and 40° C. Incubation periods are selected for optimum activity, but can also be optimized to facilitate rapid high through put screening. Typically between 0.1 and 1 hour will be sufficient. Excess reagent is generally removed or washed away. The second component is then added, and the presence or absence of the labeled component is followed, to indicate binding.

In other methods, the competitor is added first, followed by the candidate bioactive agent. Displacement of the competitor is an indication that the candidate bioactive agent is binding to the p38β MAP kinase protein and thus is capable of binding to, and potentially modulating, the activity of the protein. In this method, either component can be labeled. For example, if the competitor is labeled, the presence of label in the wash solution indicates displacement by the agent. Alternatively, if the candidate bioactive agent is labeled, the presence of the label on the support indicates displacement.

In other methods, the candidate bioactive agent is added first, with incubation and washing, followed by the competitor. The absence of binding by the competitor can indicate that the bioactive agent is bound to the p38β MAP kinase protein with a higher affinity. Thus, if the candidate bioactive agent is labeled, the presence of the label on the support, coupled with a lack of competitor binding, can indicate that the candidate agent is capable of binding to the protein.

Competitive binding methods can also be run as differential screens. These methods can comprise a p38β MAP kinase protein and a competitor in a first sample. A second sample comprises a candidate bioactive agent, a p38β MAP kinase protein and a competitor. The binding of the competitor is determined for both samples, and a change, or difference in binding between the two samples indicates the presence of an agent capable of binding to the p38β MAP kinase protein and potentially modulating its activity. If the binding of the competitor is different in the second sample relative to the first sample, the agent is capable of binding to the protein.

Other methods utilize differential screening to identify drug candidates that bind to the native p38β MAP kinase protein, but cannot bind to modified proteins. The structure of the protein can be modeled, and used in rational drug design to synthesize agents that interact with that site. Drug candidates that affect p38α MAP kinase bioactivity are also identified by screening drugs for the ability to either enhance or reduce the activity of the protein.

In some methods, screening for agents that modulate the activity of proteins are performed. In general, this will be done on the basis of the known biological activity of the p38β MAP kinase protein. In these methods, a candidate bioactive agent is added to a sample of the protein, as above, and an alteration in the biological activity of the protein is determined.

“Modulating the activity” includes an increase in activity, a decrease in activity, or a change in the type or kind of activity present. Thus, in these methods, the candidate agent should both bind to p38β MAP kinase (although this may not be necessary), and alter its biological or biochemical activity as defined herein. The methods include both in vitro screening methods, as are generally outlined above, and in vivo screening of cells for alterations in the presence, distribution, activity or amount of the protein.

Some methods comprise combining a p38β MAP kinase sample and a candidate bioactive agent, then evaluating the effect on p38β MAP kinase anti-nociceptive activity. By “p38β MAP kinase activity” or grammatical equivalents herein is meant one of p38β MAP kinase biological activities, including, but not limited to, its ability to prevent a facilitative state for sensation of pain, e.g., hyperalgesia or allodynia. One activity herein is the capability to bind to a target gene, or modulate p38β MAP kinase signaling.

In other methods, the activity of the p38β MAP kinase protein is decreased. Thus, bioactive agents that are antagonists are useful in some methods.

Methods for screening for bioactive agents capable of modulating the activity of a p38β MAP kinase protein are provided. These methods comprise adding a candidate bioactive agent, as defined above, to a cell comprising proteins. Cell types include almost any cell. The cells contain a recombinant nucleic acid that encodes a p38β MAP kinase protein. In one method, a library of candidate agents are tested on a plurality of cells. The effect of the candidate agent on p38β MAP kinase pathway signaling activity is then evaluated.

Positive controls and negative controls can be used in the assays. All control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples can be counted in a scintillation counter to determine the amount of bound compound.

A variety of other reagents can be included in the screening assays. These include reagents like salts, neutral proteins (e.g., albumin and detergents) which can be used to facilitate optimal protein-protein binding and/or reduce non-specific or background interactions. Reagents that otherwise improve the efficiency of the assay, (such as protease inhibitors, nuclease inhibitors, anti-microbial agents) can also be used. The mixture of components can be added in any order that provides for the requisite binding.

The components provided herein for the assays provided herein can also be combined to form kits. The kits can be based on the use of the protein and/or the nucleic acid encoding the p38β MAP kinase proteins. Assays regarding the use of nucleic acids are further described below.

(B) Animal Models

In one method, nucleic acids which encode p38β MAP kinase proteins or their modified forms can also be used to generate either transgenic animals, including “knock-in” and “knock out” animals which, in turn, are useful in the development and screening of therapeutically useful reagents. A non-human transgenic animal (e.g., a mouse or rat) is an animal having cells that contain a transgene, which transgene is introduced into the animal or an ancestor of the animal at a prenatal, e.g., an embryonic stage. A transgene is a DNA which is integrated into the genome of a cell from which a transgenic animal develops, and can include both the addition of all or part of a gene or the deletion of all or part of a gene. In some methods, cDNA encoding a p38β MAP kinase protein can be used to clone genomic DNA encoding a p38β MAP kinase protein in accordance with established techniques and the genomic sequences used to generate transgenic animals that contain cells which either express (or overexpress) or suppress the desired DNA. Methods for generating transgenic animals, particularly animals such as mice or rats, have become conventional in the art and are described, for example, in U.S. Pat. Nos. 4,736,866 and 4,870,009, each incorporated herein by reference in their entirety. Typically, particular cells would be targeted for a p38β MAP kinase protein transgene incorporation with tissue-specific enhancers. Transgenic animals that include a copy of a transgene encoding a p38β MAP kinase protein introduced into the germ line of the animal at an embryonic stage can be used to examine the effect of increased expression of the desired nucleic acid. Such animals can be used as tester animals for reagents thought to confer protection from, for example, pathological conditions associated with its overexpression. In accordance with this facet, an animal is treated with the reagent and a reduced incidence of the pathological condition, compared to untreated animals bearing the transgene, would indicate a potential therapeutic intervention for the pathological condition. Similarly, non-human homologues of a p38β MAP kinase protein can be used to construct a transgenic animal comprising a protein “knock out” animal which has a defective or altered gene encoding a p38β MAP kinase protein as a result of homologous recombination between the endogenous gene encoding a p38β MAP kinase protein and altered genomic DNA encoding the protein introduced into an embryonic cell of the animal. For example, cDNA encoding a p38β MAP kinase protein can be used to clone genomic DNA encoding the protein in accordance with established techniques. A portion of the genomic DNA encoding a p38β MAP kinase protein can be deleted or replaced with another gene, such as a gene encoding a selectable marker which can be used to monitor integration. Typically, several kilobases of unaltered flanking DNA (both at the 5′ and 3′ ends) are included in the vector (see, e.g., Thomas and Capecchi, Cell 51:503, 1987, incorporated herein by reference in its entirety, for a description of homologous recombination vectors). The vector is introduced into an embryonic stem cell line (e.g., by electroporation) and cells in which the introduced DNA has homologously recombined with the endogenous DNA are selected (see, e.g., Li et al., Cell 69: 915, 1992, incorporated herein by reference in its entirety). The selected cells are then injected into a blastocyst of an animal (e.g., a mouse or rat) to form aggregation chimeras (see, e.g., Bradley, in Teratocarcinomas and Embryonic Stem Cells: A Practical Approach, E. J. Robertson, ed. (IRL, Oxford, 1987), pp. 113-152). A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of a p38β MAP kinase protein polypeptide.

Animals of any species, including, but not limited to, mice, rats, rabbits, guinea pigs, pigs, micro-pigs, goats, and non-human primates, e.g., baboons, monkeys, and chimpanzees can be used to generate animal models of p38β MAP kinase pathway signaling related disorders or being a perpetually desired state of the p38α MAP kinase pathway signaling.

(C) Nucleic Acid Based Therapeutics

Nucleic acids encoding p38β MAP kinase polypeptides or p38β MAP kinase antagonists can also be used in gene therapy. Broadly speaking, a gene therapy vector is an exogenous polynucleotide which produces a medically useful phenotypic effect upon the mammalian cell(s) into which it is transferred. A vector can or can not have an origin of replication. For example, it is useful to include an origin of replication in a vector for propagation of the vector prior to administration to a patient. However, the origin of replication can often be removed before administration if the vector is designed to integrate into host chromosomal DNA or bind to host mRNA or DNA. Vectors used in gene therapy can be viral or nonviral. Viral vectors are usually introduced into a patient as components of a virus. Nonviral vectors, typically dsDNA, can be transferred as naked DNA or associated with a transfer-enhancing vehicle, such as a receptor-recognition protein, lipoamine, or cationic lipid.

Viral vectors, such as retroviruses, adenoviruses, adeno associated viruses and herpes viruses, are often made up of two components, a modified viral genome and a coat structure surrounding it (see generally Smith et al., Ann. Rev. Microbiol. 49: 807-838, 1995, incorporated herein by reference in its entirety), although sometimes viral vectors are introduced in naked form or coated with proteins other than viral proteins. Most current vectors have coat structures similar to a wildtype virus. This structure packages and protects the viral nucleic acid and provides the means to bind and enter target cells. However, the viral nucleic acid in a vector designed for gene therapy is changed in many ways. The goals of these changes are to disable growth of the virus in target cells while maintaining its ability to grow in vector form in available packaging or helper cells, to provide space within the viral genome for insertion of exogenous DNA sequences, and to incorporate new sequences that encode and enable appropriate expression of the gene of interest. Thus, vector nucleic acids generally comprise two components: essential cis-acting viral sequences for replication and packaging in a helper line and the transcription unit for the exogenous gene. Other viral functions are expressed in trans in a specific packaging or helper cell line.

Nonviral nucleic acid vectors used in gene therapy include plasmids, RNAs, antisense oligonucleotides (e.g., methylphosphonate or phosphorothiolate), polyamide nucleic acids, and yeast artificial chromosomes (YACs). Such vectors typically include an expression cassette for expressing a protein or RNA. The promoter in such an expression cassette can be constitutive, cell type-specific, stage-specific, and/or modulatable (e.g., by hormones such as glucocorticoids; MMTV promoter). Transcription can be increased by inserting an enhancer sequence into the vector. Enhancers are cis-acting sequences of between 10 to 300 bp that increase transcription by a promoter. Enhancers can effectively increase transcription when either 5′ or 3′ to the transcription unit. They are also effective if located within an intron or within the coding sequence itself. Typically, viral enhancers are used, including SV40 enhancers, cytomegalovirus enhancers, polyoma enhancers, and adenovirus enhancers. Enhancer sequences from mammalian systems are also commonly used, such as the mouse immunoglobulin heavy chain enhancer.

Gene therapy vectors can be delivered in vivo by administration to an individual patient, typically by systemic administration (e.g., intravenous, intraperitoneal, intramuscular, subdermal, intrathecal or intracranial infusion) or topical application. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Modulating Signaling in p38β MAP Kinase Pathway

(A) Assays for Modulators of p38β MAP kinase Pathway Signaling

In numerous embodiments of this invention, the level of p38β MAP kinase signaling will be modulated in a cell by administering to the cell, in vivo or in vitro, any of a large number of p38β MAP kinase-modulating molecules, e.g., polypeptides, antibodies, amino acids, nucleotides, lipids, carbohydrates, or any organic or inorganic molecule.

To identify molecules capable of modulating p38β MAP kinase signaling, e.g., inhibitors of p38β MAP kinase or of p38β MAP kinase signaling, assays will be performed to detect the effect of various compounds on p38β MAP kinase signaling activity in a cell. p38β MAP kinase signaling can be assessed using a variety of in vitro and in vivo assays to determine functional, chemical, and physical effects, e.g., measuring the binding of p38β MAP kinase to other molecules (e.g., radioactive binding to p38β MAP kinase), measuring protein and/or RNA levels of p38β MAP kinase signaling that protect against pain, hyperalgesia, allodynia, or nociceptive events, or measuring other aspects of pathway signaling, e.g., phosphorylation levels, transcription levels, receptor activity, ligand binding and the like. Such assays can be used to test for both activators and inhibitors of p38β MAP kinase signaling. Modulators thus identified are useful for, e.g., many diagnostic and therapeutic applications.

The p38β MAP kinase signaling in the assay will typically be a recombinant or naturally occurring polypeptide or a conservatively modified variant thereof. Alternatively, the p38β MAP kinase signaling in the assay will be derived from a eukaryote and include an amino acid subsequence having amino acid sequence identity to the naturally occurring p38β MAP kinase pathway signaling. Generally, the amino acid sequence identity will be at least 70%, optionally at least 75%, 85%, or 86%, 87%, 88%, 89%, 90%, 91%, 92%, 93%, 94%, 95%, 96%, 97%, 98% or greater. Optionally, the polypeptide of the assays will comprise a domain of an p38β MAP kinase. In certain embodiments, a domain of p38β MAP kinase protein is bound to a solid substrate and used, e.g., to isolate any molecules that can bind to and/or modulate their activity. In certain embodiments, a domain of a p38β MAP kinase polypeptide, e.g., an N-terminal domain, a C-terminal domain, is fused to a heterologous polypeptide, thereby forming a chimeric polypeptide. Such chimeric polypeptides are also useful, e.g., in assays to identify modulators of an p38β MAP kinase signaling.

The terms “identical” or percent “identity,” in the context of two or more nucleic acids or polypeptide sequences, refers to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequence encoding a collectin described herein or amino acid sequence of a collectin described herein), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site). Such sequences are then said to be “substantially identical.” This term also refers to, or can be applied to, the compliment of a test sequence. The term also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math., 1981, 2:482, by the homology alignment algorithm of Needleman & Wunsch, J. Mol. Biol., 1970, 48:443, by the search for similarity method of Pearson & Lipman, Proc. Nat'l. Acad. Sci. USA, 1988, 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology, Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res., 25: 3389-3402, 1977 and Altschul et al., J. Mol. Biol. 215: 403-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 1989, 89:10915) alignments (B) of 50, expectation (E) of 10, M=5, N=−4, and a comparison of both strands.

The terms “polypeptide”, “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins, 1984).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I: The Conformation of Biological Macromolecules, 1980. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity, e.g., a kinase domain. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript can be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions can also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., Ausubel et al, supra.

For PCR, a temperature of about 36° C. is typical for low stringency amplification, although annealing temperatures can vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al PCR Protocols, A Guide to Methods and Applications, Academic Press, Inc. N.Y. (1990).

Samples or assays that are treated with a potential p38β MAP kinase pathway signaling inhibitor or activator are compared to control samples without the test compound, to examine the extent of modulation. Control samples (untreated with activators or inhibitors) are assigned a relative p38β MAP kinase activity value of 100. Inhibition of p38β MAP kinase pathway signaling is achieved when the p38β MAP kinase pathway signaling activity value relative to the control is about 90%, optionally about 50%, optionally about 25-0%. Activation of a p38β MAP kinase pathway signaling is achieved when the p38β MAP kinase pathway signaling activity value relative to the control is about 110%, optionally about 150%, 200-500%, or about 1000-2000%.

The effects of the test compounds upon the function of the polypeptides can be measured by examining any of the parameters described above. Any suitable physiological change that affects p38β MAP kinase pathway signaling activity can be used to assess the influence of a test compound on the polypeptides of this invention. When the functional consequences are determined using intact cells or animals, one can also measure a variety of effects such as changes in cell growth or changes in cell-cell interactions.

Modulators of p38β MAP kinase pathway signaling that act by modulating gene expression can also be identified. For example, a host cell containing a p38β MAP kinase protein of interest is contacted with a test compound for a sufficient time to effect any interactions, and then the level of gene expression is measured. The amount of time to effect such interactions can be empirically determined, such as by running a time course and measuring the level of transcription as a function of time. The amount of transcription can be measured using any method known to those of skill in the art to be suitable. For example, mRNA expression of the protein of interest can be detected using Northern blots or by detecting their polypeptide products using immunoassays.

(B) Assays for Inhibitors of p38β MAP Kinase Pathway Signaling

In certain embodiments, assays will be performed to identify molecules that physically interact with p38β MAP kinase. Such molecules can be any type of molecule, including polypeptides, polynucleotides, amino acids, nucleotides, carbohydrates, lipids, or any other organic or inorganic molecule. Such molecules can represent molecules that normally interact with p38β MAP kinase or can be synthetic or other molecules that are capable of interacting with p38β MAP kinase and that can potentially be used as lead compounds to identify classes of molecules that can interact with and/or modulate or inhibit p38β MAP kinase signaling. Such assays can represent physical binding assays, such as affinity chromatography, immunoprecipitation, two-hybrid screens, or other binding assays, or can represent genetic assays.

In any of the binding or functional assays of p38β MAP kinase signaling described herein, in vivo or in vitro, any derivative, variation, homolog, or fragment of p38β MAP kinase, can be used. Preferably, the p38β MAP kinase or variant thereof has at least about 85% identity to the amino acid sequence of the naturally occurring p38β MAP kinase. In numerous embodiments, a fragment of p38β MAP kinase is used. Such fragments can be used alone, in combination with other p38β MAP kinase protein fragments, or in combination with sequences from heterologous proteins, e.g., the fragments can be fused to a heterologous polypeptides, thereby forming a chimeric polypeptide.

Compounds that interact with p38β MAP kinase signaling can be isolated based on an ability to specifically bind to p38β MAP kinase or fragment thereof. In numerous embodiments, the p38β MAP kinase or protein fragment will be attached to a solid support. In one embodiment, affinity columns are made using the p38β MAP kinase polypeptide, and physically-interacting molecules are identified. It will be apparent to one of skill that chromatographic techniques can be performed at any scale and using equipment from many different manufactures (e.g., Pharmacia Biotechnology). In addition, molecules that interact with p38β MAP kinase in vivo can be identified by co-immunoprecipitation or other methods, i.e., immunoprecipitating p38β MAP kinase using anti-p38β MAP kinase antibodies from a cell or cell extract, and identifying compounds, e.g., proteins, that are precipitated along with the p38β MAP kinase. Such methods are well known to those of skill in the art and are taught, e.g., in Ausubel et al., 1994; Sambrook et al., Molecular Cloning: A Laboratory Manual, Cold Spring Harbor Press, NY., 1989; and Harlow and Lane, Antibodies: A Laboratory Manual, Cold Spring Harbor Press, NY., 1989.

(C) Reducing p38β MAP Kinase Protein Activity Levels In Cells

In certain embodiments, this invention provides methods of preventing a facilitative state for sensation of pain, e.g., hyperalgesia or allodynia, by reducing p38β MAP kinase protein levels in a cell. Typically, such methods are used to reduce an elevated level of p38β MAP kinase protein, e.g., an elevated level in a microglial or neuronal cell or neuronal cell, and can be performed in any of a number of ways, e.g., lowering the copy number of p38β MAP kinase protein genes or decreasing the level of mRNA, protein, or protein activity in a cell. Preferably, the level of p38β MAP kinase protein activity is lowered to a level typical of a normal microglial or neuronal cell or neuronal cell, but the level can be reduced to any level that is sufficient to increase p38β MAP kinase signaling of the cell, including to levels above or below those typical of normal cells. Preferably, such methods involve the use of inhibitors of p38β MAP kinase protein, where an “inhibitor of p38β MAP kinase protein” is a molecule that acts to reduce p38β MAP kinase protein polynucleotide levels, polypeptide levels and/or protein activity. Such inhibitor s include, but are not limited to, antisense polynucleotides, ribozymes, antibodies, dominant negative p38β MAP kinase protein forms, and small molecule inhibitors of p38β MAP kinase protein.

In preferred embodiments, p38β MAP kinase protein levels will be reduced so as to prevent a facilitative state for sensation of pain, e.g., hyperalgesia or allodynia. The proliferation of a cell refers to the rate at which the cell or population of cells divides, or to the extent to which the cell or population of cells divides or increases in number. Proliferation can reflect any of a number of factors, including the rate of cell growth and division and the rate of cell death. Without being bound by the following offered theory, it is suggested that the inhibition of the p38β MAP kinase gene in microglial or neuronal cell or neuronal cells will prevent nociceptive signaling. The anti-nociceptive activity of inhibitors of p38β MAP kinase protein can to prevent a facilitative state for sensation of pain, e.g., hyperalgesia or allodynia. The ability of any of the present compounds to affect p38β MAP kinase protein activity can be determined based on any of a number of factors, including, but not limited to, a level of p38α MAP kinase polynucleotide, e.g., mRNA or gDNA, the level of p38β MAP kinase polypeptide, the degree of binding of a compound to a p38β MAP kinase polynucleotide or polypeptide, p380 MAP kinase protein intracellular localization, or any functional properties of p38β MAP kinase protein, such as the ability of p38β MAP kinase protein activity to prevent a facilitative state for sensation of pain.

(D) Inhibitors of p38β MAP Kinase Polynucleotides

In certain embodiments, p38β MAP kinase protein activity is downregulated, or entirely inhibited, by the use of antisense polynucleotide, i.e., a nucleic acid complementary to, and which can preferably hybridize specifically to, a coding mRNA nucleic acid sequence, e.g., p38β MAP kinase induced mRNA, or a subsequence thereof. Binding of the antisense polynucleotide to the mRNA reduces the translation and/or stability of the p38β MAP kinase induced mRNA.

In the context of this invention, antisense polynucleotides can comprise naturally-occurring nucleotides, or synthetic species formed from naturally-occurring subunits or their close homologs. Antisense polynucleotides can also have altered sugar moieties or inter-sugar linkages. Exemplary among these are the phosphorothioate and other sulfur containing species which are known for use in the art. All such analogs are comprehended by this invention so long as they function effectively to hybridize with p38β MAP kinase induced mRNA.

Such antisense polynucleotides can readily be synthesized using recombinant means, or can be synthesized in vitro. Equipment for such synthesis is sold by several vendors, including Applied Biosystems. The preparation of other oligonucleotides such as phosphorothioates and alkylated derivatives is also well known to those of skill in the art.

In addition to antisense polynucleotides, ribozymes can be used to target and inhibit transcription of p38β MAP kinase protein. A ribozyme is an RNA molecule that catalytically cleaves other RNA molecules. Different kinds of ribozymes have been described, including group I ribozymes, hammerhead ribozymes, hairpin ribozymes, RNAse P, and axhead ribozymes (see, e.g., Castanotto et al., Adv. in Pharmacology 25: 289-317, 1994 for a general review of the properties of different ribozymes).

The general features of hairpin ribozymes are described, e.g., in Hampel et al., Nucl. Acids Res., 18: 299-304, 1990; Hampel et al., European Patent Publication No. 0 360 257, 1990; U.S. Pat. No. 5,254,678. Methods of preparing are well known to those of skill in the art (see, e.g., Wong-Staal et al., WO 94/26877; Ojwang et al., Proc. Natl. Acad. Sci. USA, 90: 6340-6344, 1993; Yamada et al., Human Gene Therapy 1: 39-45, 1994; Leavitt et al., Proc. Natl. Acad. Sci. USA, 92: 699-703, 1995; Leavitt et al., Human Gene Therapy 5: 1151-120, 1994; and Yamada et al., Virology 205: 121-126, 1994).

p38β MAP kinase protein activity can also be decreased by the addition of an inhibitor of the p38β MAP kinase protein. This can be accomplished in any of a number of ways, including by providing a dominant negative p38β MAP kinase polypeptide, e.g., a form of p380 MAP kinase protein that itself has no activity and which, when present in the same cell as a functional p38β MAP kinase protein, reduces or eliminates the p38β MAP kinase protein activity of the functional p38β MAP kinase protein. Design of dominant negative forms is well known to those of skill and is described, e.g., in Herskowitz, Nature 329:219-22, 1987. Also, inactive polypeptide variants (muteins) can be used, e.g., by screening for the ability to inhibit p38β MAP kinase protein activity. Methods of making muteins are well known to those of skill (see, e.g., U.S. Pat. Nos. 5,486,463; 5,422,260; 5,116,943; 4,752,585; and 4,518,504). In addition, any small molecule, e.g., any peptide, amino acid, nucleotide, lipid, carbohydrate, or any other organic or inorganic molecule can be screened for the ability to bind to or inhibit p38β MAP kinase protein activity, as described below.

(E) Modulators and Binding Compounds

The compounds tested as modulators or inhibitors of a p38β MAP kinase protein can be any small chemical compound, or a biological entity, such as a protein, sugar, nucleic acid or lipid. Typically, test compounds will be small chemical molecules and peptides. Essentially any chemical compound can be used as a potential modulator or binding compound in the assays of the invention, although most often compounds can be dissolved in aqueous or organic (especially DMSO-based) solutions. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma-Aldrich (St. Louis, Mo.), Fluka Chemika-Biochemica Analytika (Buchs, Switzerland) and the like.

In one preferred embodiment, high throughput screening methods involve providing a combinatorial chemical or peptide library containing a large number of potential therapeutic compounds (potential modulator or binding compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka, Int. J. Pept. Prot. Res. 37:487-493, 1991; and Houghton et al., Nature 354: 84-88, 1991). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90: 6909-6913, 1993), vinylogous polypeptides (Hagihara et al., J. Amer. Chem. Soc. 114: 6568, 1992), nonpeptidal peptidomimetics with glucose scaffolding (Hirschmann et al., J. Amer. Chem. Soc. 114: 9217-9218, 1992), analogous organic syntheses of small compound libraries (Chen et al., J. Amer. Chem. Soc. 116: 2661, 1994), oligocarbamates (Cho et al., Science 261:1303, 1993), and/or peptidyl phosphonates (Campbell et al., J. Org. Chemn. 59: 658, 1994), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology 14: 309-314, 1996; and PCT/US96/10287), carbohydrate libraries (see, e.g., Liang et al., Science, 274:1520-1522, 1996; and U.S. Pat. No. 5,593,853), small organic molecule libraries (see, e.g., benzodiazepines, Baum, C&EN, page 33, Jan. 18, 1993; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J.; Tripos, Inc., St. Louis, Mo.; 3D Pharmaceuticals, Exton, Pa.; Martek Biosciences, Columbia, Md., etc.).

(F) Solid State and Soluble High Throughput Assays

In one embodiment, the invention provides soluble assays using molecules such as an N-terminal or C-terminal domain either alone or covalently linked to a heterologous protein to create a chimeric molecule. In another embodiment, the invention provides solid phase based in vitro assays in a high throughput format, where a domain, chimeric molecule, p38β MAP kinase protein, or cell or tissue expressing a p38β MAP kinase protein is attached to a solid phase substrate.

In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 different compounds is possible using the integrated systems of the invention. More recently, microfluidic approaches to reagent manipulation have been developed.

The molecule of interest can be bound to the solid state component, directly or indirectly, via covalent or non covalent linkage, e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85: 2149-2154, 1993 (describing solid phase synthesis of, e.g., peptides); Geysen et al., J. Immun. Meth. 102: 259-274, 1987 (describing synthesis of solid phase components on pins); Frank & Doring, Tetrahedron 44: 6031-6040, 1988 (describing synthesis of various peptide sequences on cellulose disks); Fodor et al., Science 251: 767-777, 1991; Sheldon et al., Clinical Chemistry 39: 718-719, 1993; and Kozal et al., Nature Medicine 2: 753-759, 1996 (all describing arrays of biopolymers fixed to solid substrates). Nonchemical approaches for fixing tag binders to substrates include other common methods, such as heat, cross-linking by UV radiation, and the like.

(G) Rational Drug Design Assays

Yet another assay for compounds that modulate or inhibit p38β MAP kinase protein activity involves computer assisted drug design, in which a computer system is used to generate a three-dimensional structure of a p38β MAP kinase protein based on the structural information encoded by its amino acid sequence. The input amino acid sequence interacts directly and actively with a pre-established algorithm in a computer program to yield secondary, tertiary, and quaternary structural models of the protein. The models of the protein structure are then examined to identify regions of the structure that have the ability to bind. These regions are then used to identify compounds that bind to the protein.

The three-dimensional structural model of the protein is generated by entering protein amino acid sequences of at least 10 amino acid residues or corresponding nucleic acid sequences encoding a p38β MAP kinase polypeptide into the computer system. The nucleotide sequence encoding the polypeptide, or the amino acid sequence thereof, and conservatively modified versions thereof, of the naturally occurring gene sequence. The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential modulator binding regions are identified by the computer system. Three-dimensional structures for potential modulators are generated by entering amino acid or nucleotide sequences or chemical formulas of compounds, as described above. The three-dimensional structure of the potential modulator is then compared to that of the p38β MAP kinase protein to identify compounds that bind to the protein. Binding affinity between the protein and compound is determined using energy terms to determine which compounds have an enhanced probability of binding to the protein.

Computer systems are also used to screen for mutations, polymorphic variants, alleles and interspecies homologs of p38β MAP kinase-induced genes. Such mutations can be associated with disease states or genetic traits. GeneChip™ and related technology can also be used to screen for mutations, polymorphic variants, alleles and interspecies homologs. Once the variants are identified, diagnostic assays can be used to identify patients having such mutated genes. Identification of the mutated p38β MAP kinase induced genes involves receiving input of a first nucleic acid or amino acid sequence of the naturally occurring p38β MAP kinase induced gene, respectively, and conservatively modified versions thereof. The sequence is entered into the computer system as described above. The first nucleic acid or amino acid sequence is then compared to a second nucleic acid or amino acid sequence that has substantial identity to the first sequence. The second sequence is entered into the computer system in the manner described above. Once the first and second sequences are compared, nucleotide or amino acid differences between the sequences are identified. Such sequences can represent allelic differences in various p38β MAP kinase induced genes, and mutations associated with disease states and genetic traits.

### Diagnostic Methods

In addition to assays, the creation of animal models, and nucleic acid based therapeutics, identification of important genes allows the use of these genes in diagnosis (e.g., diagnosis of cell states and abnormal cell conditions). Disorders based on mutant or variant p38β MAP kinase genes can be determined. Methods for identifying cells containing variant p38β MAP kinase genes comprising determining all or part of the sequence of at least one endogeneous genes in a cell are provided. As will be appreciated by those in the art, this can be done using any number of sequencing techniques. Methods of identifying the genotype of an individual comprising determining all or part of the sequence of at least one p38β MAP kinase gene of the individual are also provided. This is generally done in at least one tissue of the individual, and can include the evaluation of a number of tissues or different samples of the same tissue. The method can include comparing the sequence of the sequenced mutant p38β MAP kinase gene to a known p38β MAP kinase gene, i.e., a wild-type gene.

The sequence of all or part of the p38β MAP kinase gene can then be compared to the sequence of a known p38β MAP kinase gene to determine if any differences exist. This can be done using any number of known sequence identity programs, such as Bestfit, and others outlined herein. In some methods, the presence of a difference in the sequence between the p38β MAP kinase gene of the patient and the known p38β MAP kinase gene is indicative of a disease state or a propensity for a disease state, as outlined herein.

Similarly, diagnosis of microglial or neuronal or neuronal cell states can be done using the methods and compositions herein. By evaluating the gene expression profile of microglial or neuronal or neuronal cells from a patient, the microglial or neuronal or neuronal cell state can be determined. This is particularly useful to verify the action of a drug, for example an anti-nociceptive drug. Other methods comprise administering the drug to a patient and removing a cell sample, particularly of microglial or neuronal or neuronal cells, from the patient. The gene expression profile of the cell is then evaluated, as outlined herein, for example by comparing it to the expression profile from an equivalent sample from a healthy individual. In this manner, both the efficacy (i.e., whether the correct expression profile is being generated from the drug) and the dose (is the dosage correct to result in the correct expression profile) can be verified.

The present discovery relating to the role of p38β MAP kinase in nociceptive states of disease thus provides methods for inducing or maintaining differing disease states. In one method, the p38β MAP kinase proteins, and particularly p38β MAP kinase protein fragments, are useful in the study or treatment of conditions which are mediated by disease states, i.e., to diagnose, treat or prevent a facilitative state for sensation of pain, e.g., hyperalgesia or allodynia.

Methods of modulating states of pain in cells or organisms are provided. Some methods comprise administering to a cell an anti-p38β MAP kinase antibody or other agent identified herein or by the methods provided herein, that reduces or eliminates the biological activity of the endogeneous p38β MAP kinase protein. Alternatively, the methods comprise administering to a cell or organism a recombinant nucleic acid encoding a p38β MAP kinase protein or modulator including anti-sense nucleic acids. As will be appreciated by those in the art, this can be accomplished in any number of ways. In one method, the gene therapy techniques include the incorporation of the exogenous gene using enhanced homologous recombination (EHR), for example as described in PCT/US93/03868, hereby incorporated by reference in its entirety.

Methods for diagnosing a microglial or neuronal or neuronal cell activity related condition in an individual are provided. The methods comprise measuring the activity of p38β MAP kinase protein in a tissue from the individual or patient, which can include a measurement of the amount or specific activity of the protein. This activity is compared to the activity of p38β MAP kinase protein from either an unaffected second individual or from an unaffected tissue from the first individual. When these activities are different, the first individual can be at risk for a microglial or neuronal or neuronal cell activity mediated disorder.

Furthermore, nucleotide sequences encoding a p38β MAP kinase protein can also be used to construct hybridization probes for mapping the gene which encodes that p38β MAP kinase protein and for the genetic analysis of individuals with genetic disorders. The nucleotide sequences provided herein can be mapped to a chromosome and specific regions of a chromosome using known techniques, such as in situ hybridization, linkage analysis against known chromosomal markers, and hybridization screening with libraries.

### Antibodies

In some methods, the p38β MAP kinase proteins can be used to generate polyclonal and monoclonal antibodies to p38β MAP kinase proteins, which are useful as modulators or inhibitors of p38β MAP kinase or as modulators or inhibitors of p38β MAP kinase signaling pathway, as described herein. A number of immunogens are used to produce antibodies that specifically bind p38β MAP kinase polypeptides. Full-length p38β MAP kinase polypeptides are suitable immunogens. Typically, the immunogen of interest is a peptide of at least about 3 amino acids, more typically the peptide is at least 5 amino acids in length, the fragment is at least 10 amino acids in length and typically the fragment is at least 15 amino acids in length. The peptides can be coupled to a carrier protein (e.g., as a fusion protein), or are recombinantly expressed in an immunization vector. Antigenic determinants on peptides to which antibodies bind are typically 3 to 10 amino acids in length. Naturally occurring polypeptides are also used either in pure or impure form. Recombinant polypeptides are expressed in eukaryotic or prokaryotic cells and purified using standard techniques. The polypeptide, or a synthetic version thereof, is then injected into an animal capable of producing antibodies. Either monoclonal or polyclonal antibodies can be generated for subsequent use in immunoassays to measure the presence and quantity of the polypeptide.

These antibodies find use in a number of applications. For example, the p38β MAP kinase antibodies can be coupled to standard affinity chromatography columns and used to purify p38β MAP kinase proteins as further described below. The antibodies can also be used as blocking polypeptides, as outlined above, since they will specifically bind to the p38β MAP kinase protein.

The anti-p38β MAP kinase antibodies can comprise polyclonal antibodies. Methods for producing polyclonal antibodies are known to those of skill in the art. In brief, an immunogen, for example, a purified polypeptide, a polypeptide coupled to an appropriate carrier (e.g., GST and keyhole limpet hemocyanin), or a polypeptide incorporated into an immunization vector such as a recombinant vaccinia virus (see, U.S. Pat. No. 4,722,848) is mixed with an adjuvant and animals are immunized with the mixture. The animal's immune response to the immunogen preparation is monitored by taking test bleeds and determining the titer of reactivity to the polypeptide of interest. When appropriately high titers of antibody to the immunogen are obtained, blood is collected from the animal and antisera are prepared. Further fractionation of the antisera to enrich for antibodies reactive to the polypeptide is performed where desired. See, e.g., Coligan, Current Protocols in Immunology, Wiley/Greene, NY, 1991; and Harlow and Lane, supra, each incorporated herein by reference in their entirety.

Antibodies, including binding fragments and single chain recombinant versions thereof, against predetermined fragments of p38β MAP kinase proteins are raised by immunizing animals, e.g., with conjugates of the fragments with carrier proteins as described above.

The anti-p38β MAP kinase antibodies can, alternatively, be monoclonal antibodies. The monoclonal antibodies are prepared from cells secreting the desired antibody. These antibodies are screened for binding to normal or modified polypeptides, or screened for agonistic or antagonistic activity, e.g., activity mediated through the p38, MAP kinase proteins. In some instances, it is desirable to prepare monoclonal antibodies from various mammalian hosts, such as mice, rodents, primates, and humans. Description of techniques for preparing such monoclonal antibodies are found in, e.g., Stites et al. (eds.) Basic and Clinical Immunology (4th ed.) Lange Medical Publications, Los Altos, Calif., and references cited therein; Harlow and Lane, Supra; Goding, 1986; Monoclonal Antibodies: Principles and Practice (2d ed.) Academic Press, New York, N.Y.; and Kohler et al., Nature 256: 495-497, 1975, each incorporated herein by reference in their entirety. See Goding, Monoclonal Antibodies: Principles and Practice, Academic Press, 1986, pp. 59-103, incorporated herein by reference in its entirety.

Immortalized cell lines are those that fuse efficiently, support stable high level expression of antibody by the selected antibody-producing cells, and are sensitive to a medium such as HAT medium. More immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol. 133:3001, 1984; Brodeur et al., Monoclonal Antibody Production Techniques and Applications, Marcel Dekker, Inc., New York, 1987, pp. 51-63, each incorporated herein by reference in their entirety).

“Endogenous” refers a protein, nucleic acid, lipid or other component produced within the body or within cells or organs of the body of a mammalian subject or originating within cells or organs of the body of a mammalian subject.

“Exogenous” refers a protein, nucleic acid, lipid, or other component originating outside the body of a mammalian subject.

“Immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of pathological inflammation, or pain, hyperalgesia, allodynia, or nociceptive events in normal human cells or tissues. “Immune cell response” further refers to the response of immune system cells to external or internal stimuli (e.g., antigen, cytokines, chemokines, and other cells) producing biochemical changes in the immune cells that result in immune cell migration, killing of target cells, phagocytosis, production of antibodies, other soluble effectors of the immune response, and the like.

“Signal transduction pathway” or “signal transduction event” refers to at least one biochemical reaction, but more commonly a series of biochemical reactions, which result from interaction of a cell with a stimulatory compound or agent. Thus, the interaction of a stimulatory compound with a cell generates a “signal” that is transmitted through the signal transduction pathway, ultimately resulting in a cellular response, e.g., an anti-nociceptive response described above.

Other suitable techniques involve selection of libraries of recombinant antibodies in phage or similar vectors. See, Huse et al., Science 246: 1275-1281, 1989; and Ward, et al., Nature 341: 544-546, 1989, each incorporated herein by reference in their entirety. Also, recombinant immunoglobulins can be produced. See, U.S. Pat. No. 4,816,567; and Queen et al., Proc. Nat'l Acad. Sci. USA 86: 10029-10033, 1989, each incorporated herein by reference in their entirety. See Winnacker, From Genes to Clones, VCH Publishers, N.Y., 1987, incorporated herein by reference in its entirety.

The vectors containing the polynucleotide sequences of interest (e.g., the heavy and light chain encoding sequences and expression control sequences) can be transferred into the host cell. Calcium chloride transfection is commonly utilized for prokaryotic cells, whereas calcium phosphate treatment or electroporation can be used for other cellular hosts. See generally Sambrook et al., Molecular Cloning: A Laboratory Manual, Cold Spring Harbor Press, 2d ed., 1989, incorporated herein by reference in its entirety. When heavy and light chains are cloned on separate expression vectors, the vectors are co-transfected to obtain expression and assembly of intact immunoglobulins. After introduction of recombinant DNA, cell lines expressing immunoglobulin products are cell selected. Cell lines capable of stable expression are useful (i.e., undiminished levels of expression after fifty passages of the cell line). See generally Scopes, Protein Purification, Springer-Verlag, N.Y., 1982, incorporated herein by reference in its entirety. Substantially pure immunoglobulins are of at least about 90 to 95% homogeneity, and are typically 98 to 99% homogeneity or more.

Frequently, the polypeptides and antibodies will be labeled by joining, either covalently or non-covalently, a substance which provides for a detectable signal. A wide variety of labels and conjugation techniques are known and are reported extensively in both the scientific and patent literature. Thus, an antibody used for detecting an analyte can be directly labeled with a detectable moiety, or can be indirectly labeled by, for example, binding to the antibody a secondary antibody that is, itself directly or indirectly labeled.

Antibodies are also used for affinity chromatography in isolating p38β MAP kinase proteins. Columns are prepared, e.g., with the antibodies linked to a solid support, e.g., particles, such as agarose, Sephadex, or the like, where a cell lysate is passed through the column, washed, and treated with increasing concentrations of a mild denaturant, whereby purified p38β MAP kinase polypeptides are released.

The protocol described by Huse is rendered more efficient in combination with phage-display technology. See, e.g., Dower et al., WO 91/17271; McCafferty et al., WO 92/01047; and U.S. Pat. Nos. 5,871,907; 5,858,657; 5,837,242; 5,733,743; and 5,565,332, each incorporated herein by reference in their entirety. In these methods, libraries of phage are produced in which members (display packages) display different antibodies on their outer surfaces. Antibodies are usually displayed as Fv or Fab fragments. Phage displaying antibodies with a desired specificity can be selected by affinity enrichment to the antigen or fragment thereof. Phage display combined with immunized transgenic non-human animals expressing human immunoglobulin genes can be used to obtain antigen specific antibodies even when the immune response to the antigen is weak.

In a variation of the phage-display method, human antibodies having the binding specificity of a selected murine antibody can be produced. See, for example, WO 92/20791, incorporated herein by reference in its entirety.

In another embodiment, fragments of antibodies against p38α MAP kinase protein or protein analogs are provided. Typically, these fragments exhibit specific binding to the p38β MAP kinase protein receptor similar to that of a complete immunoglobulin. Antibody fragments include separate heavy chains, light chains Fab, Fab′ F(ab′)₂ and Fv. Fragments are produced by recombinant DNA techniques, or by enzymic or chemical separation of intact immunoglobulins.

The antibodies can be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

An alternative approach is the generation of humanized immunoglobulins by linking the CDR regions of non-human antibodies to human constant regions by recombinant DNA techniques. See U.S. Pat. No. 5,585,089, incorporated herein by reference in its entirety. Humanized forms of non-human (e.g., murine) antibodies are immunoglobulins, immunoglobulin chains or fragments thereof (such as Fv, Fab, Fab′, Fab2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an Fc region, typically that of a human immunoglobulin. See Jones et al., Nature 321: 522-525, 1986; Riechmann et al., Nature 332: 323-329, 1988; and Presta, Curr. Op. Struct. Biol., 2: 593-596, 1992, each incorporated herein by reference in their entirety.

Chimeric and humanized antibodies have the same or similar binding specificity and affinity as a mouse or other nonhuman antibody that provides the starting material for construction of a chimeric or humanized antibody. Chimeric antibodies are antibodies whose light and heavy chain genes have been constructed, typically by genetic engineering, from immunoglobulin gene segments belonging to different species. For example, the variable (V) segments of the genes from a mouse monoclonal antibody can be joined to human constant (C) segments, such as IgG1 and IgG4. Human isotype IgG1 is typically used. A typical chimeric antibody is thus a hybrid protein consisting of the V or antigen-binding domain from a mouse antibody and the C or effector domain from a human antibody.

Humanized antibodies have variable region framework residues substantially from a human antibody (termed an acceptor antibody) and complementarity determining regions substantially from a mouse-antibody (referred to as the donor immunoglobulin). See, Queen et al., Proc. Natl. Acad. Sci. U.S.A. 86: 10029-10033, 1989; and WO 90/07861; U.S. Pat. No. 5,693,762; U.S. Pat. No. 5,693,761; U.S. Pat. No. 5,585,089; U.S. Pat. No. 5,530,101; and U.S. Pat. No. 5,225,539, each incorporated herein by reference in their entirety. The constant region(s), if present, are also substantially or entirely from a human immunoglobulin. The human variable domains are usually chosen from human antibodies whose framework sequences exhibit a high degree of sequence identity with the murine variable region domains from which the CDRs were derived. The heavy and light chain variable region framework residues can be derived from the same or different human antibody sequences. The human antibody sequences can be the sequences of naturally occurring human antibodies or can be consensus sequences of several human antibodies. See WO 92/22653, incorporated herein by reference in its entirety. Certain amino acids from the human variable region framework residues are selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

Bispecific antibodies are monoclonal, typically human or humanized, antibodies that have binding specificities for at least two different antigens. In the present case, one of the binding specificities is for the p38β MAP kinase protein, the other one is for any other antigen, and for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities Milstein and Cuello, Nature 305: 537-539, 1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J. 10: 3655-3659, 1991. Each citation incorporated herein by reference in their entirety.

The anti-p38β MAP kinase antibodies have various utilities. For example, anti-p38, MAP kinase antibodies can be used in diagnostic assays for a p38β MAP kinase protein, e.g., detecting its expression in specific cells, tissues, or serum. Various diagnostic assay techniques can be used, such as competitive binding assays, direct or indirect sandwich assays and immunoprecipitation assays conducted in either heterogeneous or homogeneous phases (Zola, Monoclonal Antibodies: A Manual of Techniques, CRC Press, Inc., 1987, pp. 147-158,). The antibodies used in the diagnostic assays can be labeled with a detectable moiety. The detectable moiety should be capable of producing, either directly or indirectly, a detectable signal. For example, the detectable moiety can be a radioisotope, such as 3H, 14C, 32P, 35S, or 125I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety can be employed, including those methods described by Hunter et al., Nature 144: 945, 1962; David et al., Biochemistry 13: 1014, 1974; Pain et al., J. Immunol. Meth. 40: 219, 1981; and Nygren, J. Histochem. and Cytochem. 30: 407, 1982. Each citation is incorporated herein by reference in their entirety.

### Pharmaceutical Compositions and Methods of Administration

Anti-p38β MAP kinase antibodies, antisense nucleic acids, or small molecule inhibitors of p38β MAP kinase can also be used in treatment. In some methods, the genes encoding the antibodies are provided, such that the antibodies bind to and modulate the p38β MAP kinase protein within the cell. In other methods, a therapeutically effective amount of an antagonist of p38β MAP kinase protein is administered to a patient. A “therapeutically effective amount”, “pharmacologically acceptable dose”, “pharmacologically acceptable amount” means that a sufficient amount of an anti-nociceptive agent or combination of agents is present to achieve a desired result, e.g., preventing, delaying, inhibiting or reversing a symptom of a disease or disorder or the progression of disease or disorder when administered in an appropriate regime.

Pharmaceutically acceptable carriers are determined in part by the particular composition being administered, as well as by the particular method used to administer the composition. Accordingly, there is a wide variety of suitable formulations of pharmaceutical compositions (see, e.g., Alfonso R Gennaro (ed), Remington: The Science and Practice of Pharmacy, (Formerly Remington's Pharmaceutical Sciences) 20th ed., Lippincott, Williams & Wilkins, 2003, incorporated herein by reference in its entirety). The pharmaceutical compositions generally comprise p38β MAP kinase protein or antagonist in a form suitable for administration to a patient. The pharmaceutical compositions are generally formulated as sterile, substantially isotonic and in full compliance with all Good Manufacturing Practice (GMP) regulations of the U.S. Food and Drug Administration.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art.

In some methods, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like, particularly the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The nucleic acids, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the packaged nucleic acid with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Compositions can be administered, for example, by intravenous infusion, orally, topically, intraperitoneally, intravesically or intrathecally. Formulations for injection can be presented in unit dosage form, e.g., in ampules or in multidose containers, with an added preservative.

Intrathecal administration allows the local administration of a compound to those regions of the spinal cord, such as to the dorsal horn regions, where polysynaptic relay of pain sensation occurs. Intrathecal administration, either via a bolus dosage or a constant infusion, delivers the compound directly to the subarachnoid space containing the cerebral spinal fluid (CSF).

Central delivery to spinal cord regions also can be effected by epidural injection to a region of the spinal cord exterior to the arachnoid membrane. It may be advantageous to add a means for enhancing permeation of the active compound through meningeal membranes. Such means are known in the art and include, but are not limited to, liposomal encapsulation, and the addition of a surfactant or an ion-pairing agent. Alternatively or additionally, increased arachnoid membrane permeation can be effected by administering a hypertonic dosing solution that increases permeability of meningeal barriers.

Administration by slow infusion is particularly useful when central routes such as intrathecal or epidural methods are employed. A number of implantable or body-mountable pumps useful in delivering compound at a regulated rate are known in the art. See, e.g., U.S. Pat. No. 4,619,652.

Injection solutions and suspensions can be prepared from sterile powders, granules, and tablets of the kind previously described. Cells transduced by the packaged nucleic acid as described above in the context of ex vivo therapy can also be administered intravenously or parenterally as described above.

The dose administered to a patient should be sufficient to effect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

In determining the effective amount of the vector to be administered in the treatment or prophylaxis of conditions resulting from expression of the p38β MAP kinase proteins of the methods and compositions, the physician evaluates circulating plasma levels of the vector, vector toxicities, progression of the disease, and the production of anti-vector antibodies. In general, the dose equivalent of a naked nucleic acid from a vector is from about 1 μg to 100 μg for a typical 70 kilogram patient, and doses of vectors which include a retroviral particle are calculated to yield an equivalent amount of therapeutic nucleic acid.

For administration, inhibitors and transduced cells can be administered at a rate determined by the LD50 of the inhibitor, vector, or transduced cell type, and the side-effects of the inhibitor, vector or cell type at various concentrations, as applied to the mass and overall health of the patient. Administration can be accomplished via single or divided doses.

Transduced cells are prepared for reinfusion according to established methods. See Abrahamsen et al., J. Clin. Apheresis 6: 48-53, 1991; Carter et al., J. Clin. Arpheresis 4: 113-117, 1998; Aebersold et al., J. Immunol. Meth. 112: 1-7, 1998; Muul et al., J. Immunol. Methods, 101: 171-181, 1987; and Carter et al., Transfusion 27: 362-365, 1987, each incorporated herein by reference in their entirety. After a period of about 2-4 weeks in culture, the cells should number between 1×108 and 1×1012. In this regard, the growth characteristics of cells vary from patient to patient and from cell type to cell type. About 72 hours prior to reinfusion of the transduced cells, an aliquot is taken for analysis of phenotype, and percentage of cells expressing the therapeutic agent.

### Kits

The p38α MAP kinase protein, antagonists of p38β MAP kinase or their homologs are useful tools for examining expression and regulation of signaling in microglial or neuronal or neuronal cells via the p38β MAP kinase signaling pathway. Reagents that specifically hybridize to nucleic acids encoding p38β MAP kinase proteins (including probes and primers of the proteins), and reagents that specifically bind to the proteins, e.g., antibodies, are used to examine expression and regulation.

Nucleic acid assays for the presence of p38β MAP kinase proteins in a sample include numerous techniques are known to those skilled in the art, such as Southern analysis, northern analysis, dot blots, RNase protection, S1 analysis, amplification techniques such as PCR and LCR, high density oligonucleotide array analysis, and in situ hybridization. In in situ hybridization, for example, the target nucleic acid is liberated from its cellular surroundings in such as to be available for hybridization within the cell while preserving the cellular morphology for subsequent interpretation and analysis. The following articles provide an overview of the art of in situ hybridization: Singer et al., Biotechniques 4: 230-250, 1986; Haase et al., Methods in Virology, VII: 189-226, 1984; and Nucleic Acid Hybridization: A Practical Approach (Hames et al., eds. 1987), each incorporated herein by reference in their entirety. In addition, p38β MAP kinase protein can be detected with the various immunoassay techniques described above. The test sample is typically compared to both a positive control (e.g., a sample expressing recombinant p38β MAP kinase protein) and a negative control.

Kits for screening microglial or neuronal cell activity modulators. Such kits can be prepared from readily available materials and reagents are provided. For example, such kits can comprise any one or more of the following materials: the p38β MAP kinase proteins, or antagonists of p38β MAP kinase, reaction tubes, and instructions for testing the activities of p38β MAP kinase genes. A wide variety of kits and components can be prepared depending upon the intended user of the kit and the particular needs of the user. For example, the kit can be tailored for in vitro or in vivo assays for measuring the activity of p38β MAP kinase proteins or microglial or neuronal cell activity modulators.

Kits comprising probe arrays as described above are provided. Optional additional components of the kit include, for example, other restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions.

Usually, the kits also contain instructions for carrying out the methods.

Other embodiments and uses will be apparent to one skilled in the art in light of the present disclosures.

### EXAMPLES

### Example 1

### p38α and p38β is Expressed in Rat Spinal Cord

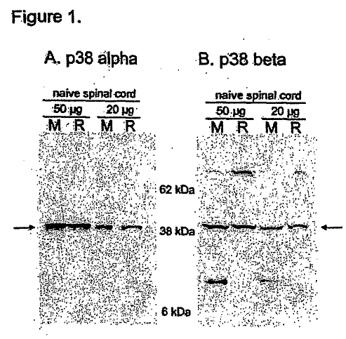

Both p38α MAP kinase and p38β MAP kinase protein expression were detected in rat lumbar spinal cord homogenates by Western blotting (FIG. 1). A single immunoreactive band was detected at about 40 kDa with the p38α antibody (FIG. 1A). Four different p38β antibodies (from Santa Cruz Biotechnology, Santa Cruz, Calif. and Zymed, San Francisco, Calif.) were tested in order to identify the antibody that gave the best signal. In our setting the monoclonal mouse anti-p38β antibody from Zymed provided the best result. This antibody, however, has been characterized for mouse, but not rat, p38β. To confirm that the antibody cross reacts with rat p38β mouse and rat naive spinal cord samples were run concurrently. A band with an approximate molecular size of 40 kDa was detected, over a range of protein concentrations for both species (FIG. 1B), supporting the presence of p38α in rat spinal cord. Additional bands were detected in both mouse and rat tissue; a lighter band (20 kDa) was detected in the mouse tissue and a heavier band (80 kDa) in the rat tissue.

FIG. 1 shows protein expression of p38α MAP kinase and p38β MAP kinase in naive rat spinal cord. (A) Western blot showing immunoreactive bands at approximately 40 kDa (arrow) indicating that p38α is expressed in naïve mouse (M) and rat (R) spinal cord (50 and 20 μg total protein loaded). (B) Western blot indicating expression of p38, (arrow) in naive mouse and rat spinal cord. Mouse and rat spinal cord samples were run at the same gel to confirm that the p38, antibody cross-reacts with rat p38β.

### Example 2

### p38α is Expressed in Neurons while p38β is Primarily Expressed in Microglia

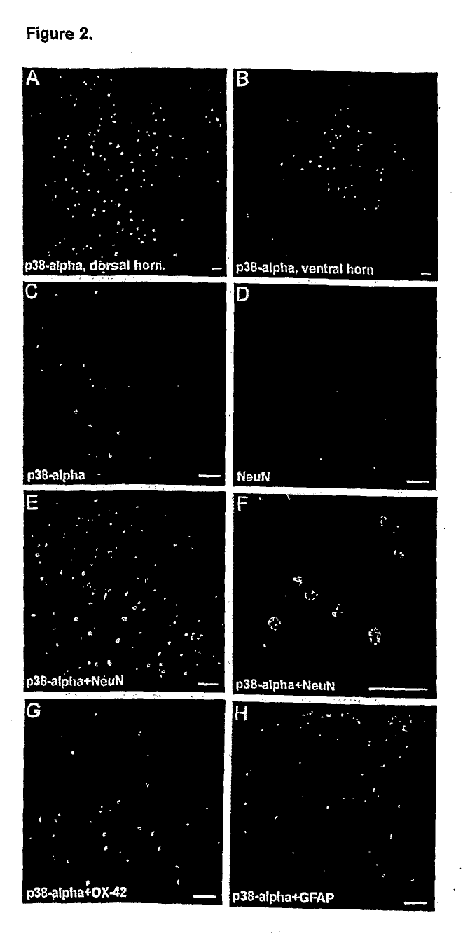

In order to determine the cellular distribution of p38α MAP kinase and/or p38β MAP kinase immunohistochemistry was undertaken. Spinal cord sections from naive animals were incubated with antibodies against p38α and p38β as well as cellular markers for neurons, astrocytes and microglia. p38α co-localized with the neuronal marker NeuN throughout the dorsal and ventral spinal parenchyma. However, the staining was weaker in laminae I and II in the dorsal horn in comparison to deeper laminae or the ventral horn (FIG. 2A-C, E). p38α does not appear to be expressed in astrocytes, as no colocalization could be detected between this isoform and GFAP (FIG. 2H). p38α co-localized with very few microglia (OX-42) in naive rat spinal cord (FIG. 2G).

FIG. 2 shows distribution and cellular localization of p38α MAP kinase in naive rat spinal cord. (A) Images depicting p38α immunoreactivity distributed throughout the dorsal horn parenchyma. The signal intensity is higher in deeper laminae than in superficial laminae. (B) Image showing presence of p38α immunoreactivity in the ventral horn of naïve rat spinal cord (arrows; motor neurons like cells, arrowheads; microglia like cells). (C-F) Double-immunofluorescence micrographs showing that spinal p38α (C, green) and the neuronal marker NeuN (D, red) are colocalized in the dorsal horn (E, yellow), indicating that p38α is expressed in neurons. Close up of p38α containing neurons shown in (F). (G) Double-labeling with antibodies against p38α (green) and the microglia marker OX-42 (red) showed no overlap indicating that p38α is not expressed in microglia. (H) Double-immunofluorescence staining p38α (green) and GFAP (red) showed no overlap, indicating that p38α is not expressed astrocytes. Size bars represent 50 μm. Similar results were observed in spinal cord tissue from a total of four rats.

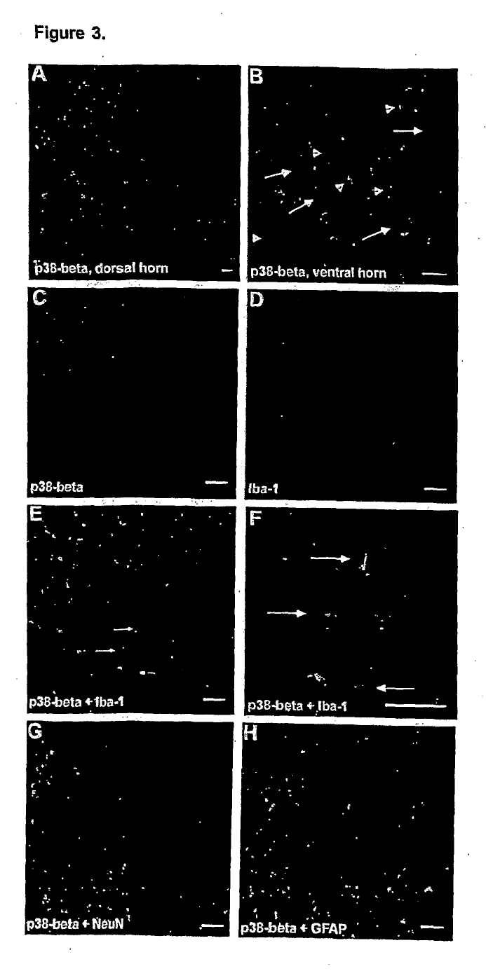

p38β was distributed homogenously throughout the spinal parenchyma (FIG. 3A-B). In contrast to the neuronal distribution of p38α, p38-β was co-localized primarily with the microglia marker Iba-1 in the dorsal horn (FIG. 3 C-F). However, in the ventral horn p38β was also noted in motor neurons as indicated in FIG. 3B. In the dorsal horn p38β was not present in cells positive for NeuN (FIG. 3G) or GFAP (FIG. 3H), indicating that p38β is not expressed in dorsal horn neurons or astrocytes.

FIG. 3 shows distribution and cellular localization of p38β MAP kinase in naive rat spinal cord. (A) Micrograph depicting p38β expression throughout the parenchyma of dorsal horn of naïve rat spinal cord. (B) Micrograph indicating presence of p38β in glia like cells as well as large motor neurons in rat naive rat ventral horn. (C-F) Double-immunofluorescence micrographs showing that antibodies against p38β (C, green) and the microglia marker Iba-1 (D, red) labels the same cell (E), indicating that p38β is expressed in microglia. Close up of p38β containing microglia shown in (F). (G) Immunofluorescence double-labeling of p38α (green) and the astrocyte marker GFAP (red) showing no overlap in the dorsal horn, indicating that p38β is not expressed in astrocytes. (H) Co-labeling of p3β (green) and Neuronal N (red) showed overlap in the ventral horn (data not shown) but not dorsal horn. Size bars represent 50 μm. Similar results were observed in spinal cord tissue from a total of four rats.

### Example 3

### Intrathecal Antisense Oligonucleotides for p38α Map Kinase and p38β Map Kinase Suppress Protein Expression of Respective p38 Isoform

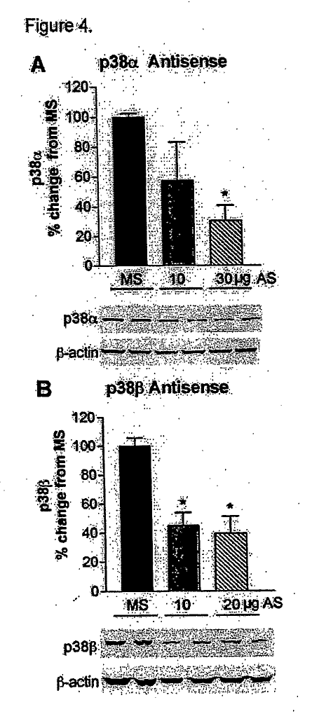

It has previously been shown that inhibition of spinal p38 MAP kinase prevents both inflammation-evoked hyperalgesia and nerve injury-induced allodynia. Ji et al., Neuron 36: 57-68, 2002; Schafers et al., J Neurosci 23: 2517-2521, 2003; Svensson et al., Neuroreport 14: 1153-1157, 2003a.; Svensson et al., J Neurochem 86: 1534-1544, 2003b. It still remains to be determined whether only one or both of the p38 isoforms, that are present in the spinal cord, are involved in modulation of spinal nociceptive processing. Since there is no specific p38 isoform inhibitors available, rats were treated intrathecally with isoform specific antisense oligonucleotides (AS) in order to knock down p38α and p38β respectively. After five days of IT bolus injections with p38α AS (10-30 μg) or p38β AS (10-20 μg) there was a significant down-regulation of both spinal p38α (FIG. 4A, 30 μg: P<0.05) and p38β (FIG. 4B, 10 and 20 μg: P<0.05) protein expression as compared to the respective protein expression in the control missense (MS)-treated animals. There were no differences between the AS-, MS- or vehicle-treated groups in terms of weight gain or motor function. The specificity of the effects of these two antisense oligonucleotides was also examined. Western blot membranes were stripped and re-incubated the with the other p38 isoform antibody. As shown in Table 1, neither p38α AS nor p38β AS produced a reduction of the protein expression of the other isoform, although p38αprotein level was somewhat increased in spinal cords of rats that had received p38β AS, as compared with MS treated rats. In Table 1, antisense was given IT once daily for 5 days and protein expression assayed in spinal chord at day six compared to protein level detected in missense treated animals (MS=100%). Each group represents mean ±SEM, n=6 per group. (*) represents P<0.05 as compared to the missense group.

TABLE 1
Protein expression after IT antisense

| | | % Expression |
|---|---|---|
| Protein | | Compared to |
| Expression | IT antisense | Missense |
| p38α | α-AS (10 μg) | 59 ± 26 |
| | α-AS (30 μg) | 39 ± 8* |
| | β-AS (20 μg) | 205 ± 65* |
| p38β | β-AS (10 μg) | 45 ± 9* |
| | β-AS (20 μg) | 39 ± 12* |
| | α-AS (30 μg) | 111 ± 15 |

*represents P < 0.05 as compared to the missense group.

FIG. 4 shows effect of IT delivery of p38 isoform specific AS on spinal p38α and p38β protein expression. (A-B) Graphs displaying p38α protein expression as % change from levels assessed in spinal cords of IT missense (MS) treated rats. Two doses of p38α AS (A, 10 and 30 μg) and p38β AS (B, 10 and 20 μg) was administrated IT for 5 days which led to a statistical significant down regulation of p38α AS p38α respectively * indicates P<0.05 as compared to MS. Representative western blots are shown below graphs. Data was normalized against β-actin protein expression in each sample and a universal MS oligonucleotide was used as a control. Each bar represents mean ±SEM, n=6 rats per group.

### Example 4

### Reduction of Spinal p38β, but not p38α, Protein Expression Suppresses Formalin-Induced Flinching

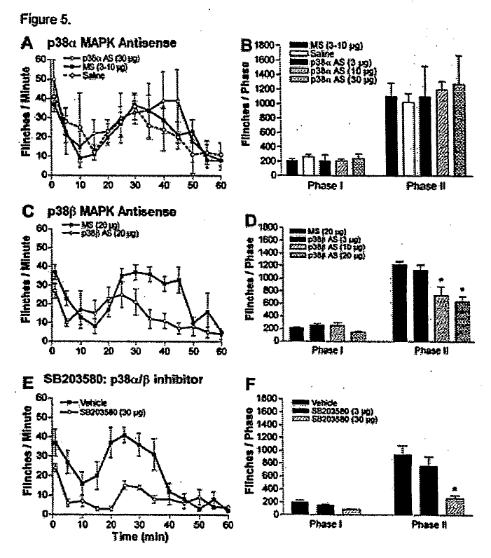

In recent work it was shown that inhibition of spinal p38 MAP kinase attenuated formalin-induced flinching. Svensson et al., J Neurochem 86: 1534-1544, 2003b. To examine the contribution of p38α and p38β in this experimental model of hyperalgesia, animals were injected with p38α, p38β AS or MS IT for five days. These animals were then examined on the formalin test. Unilateral intraplantar formalin solution (2.5%) caused a biphasic flinching pattern where the first 9 minutes are referred to as phase I and the following 50 minutes (min 10-60) as phase II. Yaksh et al., J Neurosci 21: 5847-5853, 2001a. The phase II flinching is considered to represent afferent input initiated spinal facilitation. Dickenson and Sullivan, Neurosci Lett 83: 207-211, 1987; Puig and Sorkin, Pain 64: 345-355, 1996. The total number of flinches during phase I or II recorded for animals treated IT with p38α AS did not differ from the animals treated with IT saline or MS (FIGS. 5A, B), but rather displayed a tendency to flinch more frequently. In contrast, rats that received p38β AS displayed a statistical significant reduction in number of flinches during phase two (FIGS. 5C, D) as compared to saline and MS treated animals (P<0.05). Rats that received IT-MS did not differ in number of flinches from IT-saline injected rats (FIGS. 5A, B). To further confirm the role of p38 to injury-induced pain behavior, a pharmacological inhibitor was also studied: SB203580, a p38α/β non-selective inhibitor. IT injection of SB203580 (3-30 μg) 10 minutes prior to formalin injection to the paw resulted in a dose-dependent attenuation of flinching during phase II (FIGS. 5E, F, 30 μg: P<0.05).

FIG. 5 shows effect of IT p38α and p38β AS and SB203580, a p38 inhibitor, on formalin-induced hyperalgesia. (A) Graph showing number of flinches/minute plotted versus time following injection of formalin (2.5%, 50 μl) into the dorsal side of the right hind paw in rats treated with IT p38α AS (30 ag), MS (3-10 μg) or saline daily for 5 days. (B) Bar graph showing cumulative number of flinches after paw injection of formalin during phase I (0-9 min) and phase II (10-60 min) in rats treated with different doses of IT p38α AS (3-30 μg×5 days), MS (3-10 μg) or saline daily for 5 days. There was no statistical difference between the different groups indicating that down-regulation of p38α expression does not affect formalin-induced flinching. (C) Graph showing number of flinches/minute over time following injection of formalin into the paw in rats treated with IT p38β AS (20 ag), MS (20 μg) or saline daily for 5 days. (D) Bar graph showing cumulative number of flinches after paw injection of formalin during phase I and phase II after IT injection of p38β AS (3-20 μg), MS (20 μg) or saline daily for 5 days. The number of flinches in the p38β AS (10 and 20 μg) were statistically different as compared to the MS injected group suggesting that down-regulation of p38β attenuates formalin-induced flinching. (E) Graph showing number of flinches/minute over time following injection of formalin into the paw in rats treated with IT SB203580 (30 μg), an p38β/p38α inhibitor, or vehicle 10 minutes prior to formalin injection. (F) Bar graph showing cumulative number of flinches after paw injection of formalin during phase I and phase II after IT injection of SB203580 (3-30 μg) or vehicle 10 minutes prior to formalin injection. The number of flinches in the SB203580 group (30 μg) was statistically different as compared to the vehicle group suggesting that inhibition of p38 blocks formalin-induced flinching. (*) Represents P<0.5 versus missense (MS) (D) and vehicle (F). Each time point and bar represents mean ±SEM, n=6-8 for each group.

### Example 5

### Down-Regulation of p38β, but not p38α, Blocks Formalin-Induced Phosphorylation of Spinal p38

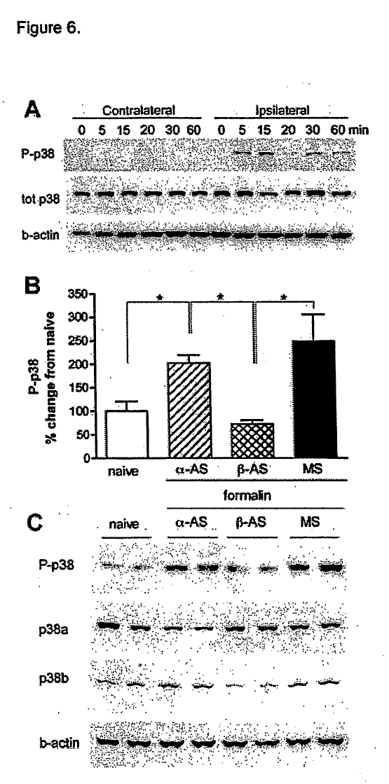

Injection of formalin into the hind paw leads to phosphorylation of p38 MAP kinase in spinal cord, by 5 minutes after injection. Svensson et al., J Neurochem 86: 1534-1544, 2003b. A full time course was performed (0, 5, 15, 20, 30, 60 min) for formalin-induced phosphorylation of spinal p38 and found peak p38 phosphorylation at 15 min (FIG. 6A). As shown above, down regulation of spinal p380, leads to attenuation of formalin-induced hyperalgesia, it was hypothesized that down-regulation of spinal p38, would reduce the amount of phosphorylated p38 in the spinal cord after formalin injection. As shown in FIGS. 6B and C, IT treatment with p38β AS lead to a reduction of phosphorylated p38 in the spinal cord, (assessed 15 minutes after formalin injection) in comparison to MS treated rats. Down-regulation of p38α AS did not prevent spinal p38 phosphorylation after paw injection of formalin as indicated by the lack of difference in spinal p38 phosphorylation between rats injected with IT-p38α AS and MS in this model (FIGS. 6B, C).

FIG. 6 shows effect of IT p38 isoform specific AS on phosphorylation of spinal p38 evoked by injection of formalin to the hind paw. (A) Representative western blots showing levels of P-p38, total p38 and β-actin in the contralateral (left side) and ipsilateral (right side) lumbar spinal cord at different time points after injection of formalin (2.5%, 50 μl) into the dorsal side of the right hind paw. (B) Rats were naïve (no IT treatment, no formalin paw injection) or treated with p38α AS (30 μg), p38β AS (20 μg) or missense (30 μg) for 5 days. On day six, formalin was injected to the hind paw and the level of phosphorylated p38 (P-p38) assessed 15 minutes after injection. The graph display % change in spinal P-p38 as compared to naïve spinal cord. Data was normalized against β-actin protein expression in each sample. Each time point and bar represents mean ±SEM, n=5 for each group. * P values <0.05. (C) Representative western blots depicting levels of spinal P-p38, p38α AS, p38, AS and β-actin 15 min after formalin injection to the paw.

### Example 6

### Down-Regulation of p38β, but not p38α, Prevents Hyperalgesia and p38 Phosphorylation Induced by Intrathecal Substance P(SP)

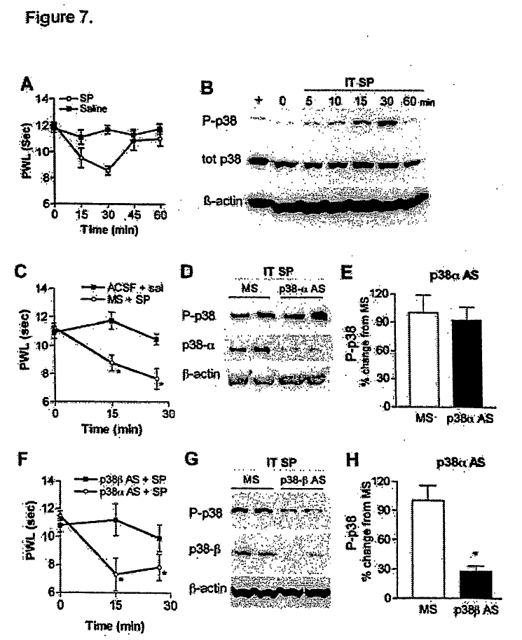

Hyperalgesia is associated with persistent sensory afferent input-induced spinal sensitization. substance P(SP), which is released from central terminals of afferent C-fibers upon stimulation contributes significantly to this facilitatory state in spinal cord. Yaksh et al., Nature 286: 155-157, 1980; Yaksh, Trends Pharmacol Sci 20: 329-337, 1999. It was shown that intrathecal injection of SP (30 nmol) produces thermal hyperalgesia and that inhibition of spinal p38α and β by IT SD-282, reverses this hyperalgesia. Malmberg and Yaksh, Science 257: 1276-1279, 1992; Svensson et al., J Neurochem 86: 1534-1544, 2003. In the present study, it was demonstrated that in parallel with development of thermal hyperalgesia, there is also a time-dependent phosphorylation of spinal p38 in a temporal pattern similar to the thermal hyperalgesia induced by IT SP, i.e., it started at 5-15 min with a peak effect at 30 min (224±41% increase as compared to saline group, N=6, P<0.05), and returned to baseline in 60 min (FIG. 7B). To investigate the contribution of p38α and p38β to SP-evoked hyperalgesia and p38 phosphorylation, spinal delivery of p38α and p38β AS and MS was carried out. Thermal withdrawal latencies was assessed after IT injection of SP and lumbar spinal cords collected immediately to compare the degree of spinal p38 phosphorylation at the hypothesized time of peak phosphorylation. IT injection of SP into the MS group produced an expected reduction of thermal withdrawal latency in comparison with IT saline injection into to vehicle treated animals (FIG. 7C). The hyperalgesia detected after IT injection of SP into the MS treated group was comparable with IT administration of SP in saline treated animals (FIG. 7A), indicating an absence of effect of IT MS. The rats receiving p38α AS showed a similar degree of thermal hyperalgesia after IT SP as the MS group (FIG. 7E). In contrast, IT SP-induced thermal hyperalgesia was attenuated in rats which had received p38β AS (FIG. 7E). Base line thermal thresholds did not differ between the groups (ACSF 10.9±0.4, MS11.45±0.5, p38α AS 11.49±0.3 and p38β AS10.8±0.5 sec). In accordance with the formalin study, the p38α AS treated group did not show a reduction in phosphorylation of spinal p38 following the hyperalgesic stimulation (FIG. 7D) while there was a pronounced reduction in spinal p38 phosphorylation in the p38β AS treated group, as compared to the MS group (FIG. 7F). Stripping and reprobing the western blot membranes confirmed that p38α protein expression was knocked down in p38α AS treated group (FIG. 7D) as well as p38β in the p38β AS group (FIG. 7F).

FIG. 7 shows effect of IT p38α and p38β AS on thermal hyperalgesia and spinal p38 phosphorylation evoked by IT SP. (A) Graph displaying paw withdrawal latency (PWL) plotted versus time. IT injection of SP (30 nmol) at T=0 resulted in a transient decrease in PWL while IT injection of saline had no effect on PWL. (B) Representative western blots showing levels of P-p38, total p38 and β-actin in lumbar spinal cord at different time points after IT injection of SP (30 nmol). IT-SP induced p38 phosphorylation peaks 30 minutes after SP injection. (C, E) Graph showing PWL assessed 15 and 27 min after bolus injection of IT saline or IT SP injection in rats treated with IT ACSF, MS (30 μg), p38α AS (30 fig) or p38β AS (20 μg) daily for five days. The hyperalgesia was not affected by IT p38α AS while p38β AS blocked IT SP-evoked thermal hyperalgesia. IT injection of MS or ACSF had no effect on IT SP-evoked thermal hyperalgesia. Each time point represents the mean ±SEM (n=4-5 per group). (D, F) Representative western blots showing the level of P-p38, p38α and p38β and β-actin protein expression assessed 30 minutes after IT SP injection. No difference in P-p38 was observed between MS and p38α AS group although p38-α protein expression was reduced in the p38α AS group. The level of P-p38 was significantly lower in the group treated with p38 AS compared with the MS group.

### Example 7

### Intrathecal SP-Induced PGE₂ Release is Mediated by Activation of p38

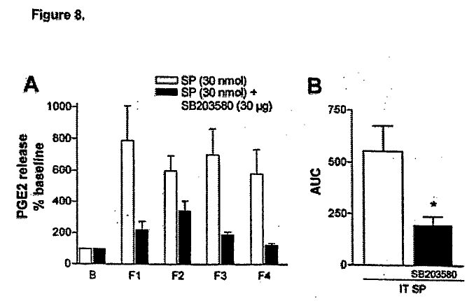

It was previously shown that intrathecal injection of SP and NMDA, but not vehicle, evokes PGE₂ release assessed by spinal dialysis. Hua et al., Neuroscience 89: 525-534, 1999b.; Svensson et al., Neuroreport 14: 1153-1157, 2003a. It has been reported that PLA₂ which generates free arachidonic acid necessary for eicosanoid synthesis, requires phosphorylation to become fully activated and that this phosphorylation can be mediated by p38 MAP kinase. Borsch-Haubold et al., Eur J Biochem 245: 751-759, 1997. The effect of spinal p38α/β inhibition was assessed on IT S P-evoked PGE₂ release by injecting SB203580 15 min prior to IT SP. Resting release of PGE₂ was determined after a washout period of 30 min. In the absence of pretreatment, baseline dialysate concentrations were determined to be 243±13 femtomol/100 μl perfusate (N=20). If SP (30 nmol) produced a 6-8 fold increase in PGE₂ level in intrathecal CSF (FIG. 8A). Pretreatment with SB203580 (30 μg, IT) (FIGS. 8A,B) prevented IT SP-evoked PGE₂ release. Comparison of AUC between groups showed that SP-evoked release of PGE₂ was significantly lower in the group that received SB203580 prior to SP (unpaired t-test P<0.05, FIG. 8B). If injection of vehicle prior to IT SP ad no effect on IT SP-evoked PGE₂ release.

FIG. 8 shows inhibition of spinal p38α/β attenuates SP-evoked spinal PGE2 release. (A) Bar graph showing PGE₂ concentration (fmol/100 μl) measured in cerebrospinal fluid collected by in vivo spinal dialysis of conscious rats before and after IT injection of substance P(SP, 30 nmol) and SB302580 (30 μg). Spinal dialysate is removed and assayed for PGE₂ by ELISA at baseline (B, average of two 15 min fractions collected prior to IT injection of SP) and F1: 0-15 minutes, F2: 15-30 minutes and F3: 30-45 minutes and 45-60 minutes after SP injection. Rats received IT SP or IT SB203580 (α/βp38 inhibitor) 15 minutes prior to IT SP. (B) PGE₂ release is presented as area under curve (AUC) calculated 60 minutes after IT injection of SP with or without pretreatment with p38 inhibitors. IT injection of vehicle prior to IT SP or IT injection of vehicle alone did not affect PGE₂ release (data not shown). Each bar represents the average and SEM for 4-8 rats per group and (*) represents P<0.5 versus baseline.

### Example 8

### Selective Inhibition of p38β Map Kinase, not p38α Map Kinase Prevents Pain

The present findings provide the following results. First, it has been demonstrated that p38α MAP kinase and p38β MAP kinase have different cellular location in the spinal dorsal horn. p38α is expressed in neurons while p38β is predominantly expressed in microglia. Second, selective down-regulation of spinal p38α and p38β protein expression was accomplished by intrathecal treatment with the respectively targeted antisense nucleic acid. Third, block of p38β, but not p38α protein expression, prevented the appearance of phosphorylated p38 otherwise evoked by stimuli which act through small afferent input (i.e., paw formalin injection) or by the direct activation of spinal sensitizing cascade though spinal NK-1 receptors by IT SP. This essentially complete block of phosphorylation suggests that the observed activation of spinal p38 was largely attributable to the p38β isoform. Fourth, selective down-regulation of p380, but not p38α, prevented hyperalgesia in both animal models. The significance of the present findings are not only in defining the role of p38β in the development of spinal sensitization but also in affirming the hypothesis that a non-neuronal origin of this isoform, i.e., microglia, mediates the hyperalgesic phenotype.

Intrathecal antisense for regulation of spinal p38 isoform expression. The initial identification of a functional role for spinal p38 in nociception arises from work showing its effect following IT delivery of p38α/β inhibitors at doses considerably lower than those reported to be active after systemic delivery. Jin et al., J Neurosci 23: 4017-4022, 2003; Schafers et al., J Neurosci 23: 2517-2521, 2003; Svensson et al., J Neurochem 86: 1534-1544, 2003b.; Sweitzer et al., Pain 109: 409-419, 2004. By either route, the identifying behavioral consequences are an absence of effect upon acute nociception but a pronounced suppression of nerve- and tissue-injury evoked hyperalgesia. Jin et al., J Neurosci 23: 4017-4022, 2003; Schafers et al., J Neurosci 23: 2517-2521, 2003; Svensson et al., J Neurochem 86: 1534-1544, 2003b.; Tsuda et al., Glia 45: 89-95, 2004. As reviewed, the p38α and p38β isoforms are found within the CNS and it is appropriate, particularly given their distinct cellular distribution in the dorsal horn, to consider the issue of which, if not both, were relevant to the observed facilitated nociceptive processing. Current agents targeted at p38 are able to readily distinguish p38α and p38β from the other isoforms (δ and γ), but to our knowledge, no available molecules exist which adequately distinguish the α and β isoforms. The use of transgenic animals to address this issue is precluded at present by the lethality of the p38 knockout. Adams et al., Mol Cell 6: 109-116, 2000; Mudgett et al., Proc Natl Acad Sci U S A 97: 10454-10459, 2000. Accordingly, the utility of intrathecally delivered oligonucleotide antisense was considered.

The use of IT AS has been well documented as a strategy to transiently knock down the expression of constitutively expressed spinal protein. Stone and Vulchanova, Adv Drug Deliv Rev 55: 1081-1112, 2003. In the present studies MOE-modified AS was used. Previous work with this structure has shown it to possess minimal toxicity and to be effective in down-regulating the expression of proteins including spinal kinases. Butler et al., Diabetes 51: 1028-1034, 2002; Henry et al., J Pharmacol Exp Ther 292: 468-479, 2000; Hua et al., Neuroscience 113: 99-107, 2002. The efficacy and specificity of the two individual AS employed in the present study was confirmed by the demonstration of (i) dose-dependent inhibition of protein expression; (ii) the differential regulation of two closely related proteins, i.e., p38α and p38β; (iii) the complete lack of activity of equal molar concentrations of control MS, and (iv) no sensory and motor dysfunction associated with either IT AS. Accordingly, based on the robust properties of IT AS, the observation in the present studies that only treatment with IT-p38β AS produced a uniform anti-hyperalgesic effect in two animal models as well as served to prevent the respective stimulus-induced spinal p38 phosphorylation is strong evidence supporting the role of the p38β isoform in spinal nociceptive processing.

p38α and p38β isoform in spinal cord. The preeminence of spinal p38β versus p38α in mediating hyperalgesia endows particular significance to their respective cellular expression. Here the present invention has determined that p38α is found predominantly in neurons while p38β is localized to microglia throughout the spinal parenchyma and motor neurons in the ventral horn. The absence of p38α in spinal microglia is somewhat unexpected given its reported expression in human primary cultures of monocytes and macrophages. Hale et al., J Immunol 162: 4246-4252, 1999. While p38β protein expression was observed in spinal microglia in the present experiments, Hale and colleagues could not detect p38β in either monocytes or macrophages, pointing to differences in hematopoetic cells potentially based on their location (peripheral versus central) as well as their activation state (cultured cells versus naive tissue). Hale et al., J Immunol 162: 4246-4252, 1999.

Down-regulation of p38α does not affect Hyperalgesia. Although presented data show that down-regulation of p38α does not affect hyperalgesia in our models, it does not exclude the possibility that this isoform may be involved in other models of more chronic hyperpathia. For example, p38 is known to regulate long-term adaptive changes (hours to days) in expression of proteins such as cytokines, enzymes (i.e. cyclooxygenase-2) and receptors (i.e. vanilloid receptor-1), that are important for spinal sensitization. Kumar et al., Nat Rev Drug Discov 2: 717-726, 2003; Lasa et al., Mol Cell Biol 20: 4265-4274, 2000; Ji et al., Neuron 36: 57-68, 2002. At this point it is unknown which isoform that is responsible for this long-term regulation. Also, it has been reported that p38α is involved in peripheral inflammation and this isoform might play a more important role in nociception at the local site of injury. Hale et al., J Immunol 162: 4246-4252, 1999. It was observed that there was an increase in spinal p38α expression in the rats treated with p38β AS, suggesting a compensatory up-regulation of p38α in response to the decreased expression of p38β. Since IT delivery of SB203580, an inhibitor to both isoforms with similar affinity displayed potent inhibition on formalin paw injection-induced flinches, that is parallel to the effect seen in p38β knock down animals, these experiments indicate that p38α isoform does not have acute influence on spinal nociceptive processing. Barone et al., Med Res Rev 21: 129-145, 2001.

The spinal p38 MAPK/COX/PLA₂ cascade. In the present study, it was shown that IT SP will produce a thermal hyperalgesia and a concurrent release of PGE₂. Previous work has shown that SP-evoked thermal hyperalgesia and spinal PGE₂ release is mediated by the activation of NK-1 receptors. Hua et al., Neuroscience 89: 525-534, 1999a. Additionally, PGE₂ synthesis and hyperalgesia are both reversed by spinal COX-2 inhibition. Malmberg and Yaksh, Science 257: 1276-1279, 1992; Yaksh et al., J Neurosci 21: 5847-5853, 2001. The present invention shows that the hyperalgesia was blocked in animals treated with p38β-, but not p38α AS, or by intrathecal delivery of the p38α/β non-specific inhibitor SB203580. Importantly, it was further shown that PGE₂ release evoked by IT SP is also blocked by the same dose of SB203580, which reverses the hyperalgesia. Svensson et al., Neuroreport 14: 1153-1157, 2003. The use of IT AS in defining the isoform role in PGE release was precluded by the technical necessity of having to treat the animal with IT AS for 5 days and the limited viability of long term intrathecal dialysis probes. Koetzner et al., J Neurosci In print, 2004. Nevertheless, these data jointly support the hypothesized cascade which in visions that persistent excitatory input activating a variety of dorsal horn receptors leads to activation of p38 isoforms which, through constitutively expressed dorsal horn PLA₂ and COX-2, yield an increase in extracellular PGE₂. Considerable evidence has shown the importance of local PGE (EP) receptors in facilitating dorsal horn processing through an enhanced afferent transmitter release and a direct sensitization of the post synaptic neuron. Hingtgen et al., J Neurosci 15: 5411-5419, 1995; Nicol et al., J Neurosci 12: 1917-1927, 1992; Southall and Vasko, J Biol Chem 276: 16083-16091, 2001; Vasko, Prog Brain Res 104: 367-380, 1995; Baba et al., J Neurosci 21: 1750-1756, 2001.

Microglia in nociceptive processing. The present invention demonstrates a central role for p38β in facilitated of spinal pain processing provides direct support for an equally central role of the local dorsal horn microglia. The present findings that p38β contributes to hyperalgesia and that this isoform is located in microglia, provides an important corollary to previous observations that p38 is activated in spinal cord microglia in several models of tissue and nerve injury. Ji et al., Neuron 36: 57-68, 2002; Svensson et al., J Neurochem 86: 1534-1544, 2003b; Tsuda et al., Glia 45: 89-95, 2004. While glia have typically been viewed as playing a supportive role in neuronal function, it has become increasingly clear that these cells are key in the regulation of spinal plasticity. Watkins et al., Pain 93: 201-205, 2001.

An important question is the nature of the communication between sensory neurons and microglia. Microglia express receptors for many neurotransmitters and neuromodulators and can synthesize and release neuroactive factors upon activation, including prostanoids and cytokines. Inagaki et al., Neurosci Lett 128: 257-260, 1991; Kommers et al., Neurosci Lett 248: 141-143, 1998; Palma et al., Glia 21: 183-193, 1997; Watkins and Maier, Annu Rev Psychol 51: 29-57, 2000. The present study, together with previous work clearly indicates that SP is one of the mediators delivering message from primary afferents to microglia. Svensson et al., Neuroreport 14: 1153-1157, 2003a. Previously it was demonstrated by immunocytochemistry that phosphorylated p38, observed 10 min after IT SP, is exclusively located in microglia, and this effect is mediated via activation of NK-1 receptors. Svensson et al., Neuroreport 14: 1153-1157, 2003a. Expression of functional NK-1 receptors on cultured murine microglia has been reported, although it has not yet been confirmed in rat spinal cord in vivo. Rasley et al., Glia 37: 258-267, 2003. It can be hypothesized that afferent evoked SP release will spread sensory including nociceptive signals to ruicroglia (directly via NK-1 receptors or indirectly via other proteins), leading to subsequent activation of p38β in these cells, that will contribute to spinal sensitization. Glutamate, another important neurotransmitter released from primary afferents has also been implicated in microglia-p38-mediated hyperalgesia and in spinal neuron hyperexcitability. Tikka and Koistinaho, J Immunol 166: 7527-7533, 2001; Svensson et al., J Neurochem 86: 1534-1544, 2003b. There are a number of other possibilities being explored, including the role of ATP released from afferents and shown to trigger microglial or neuronal responses through activation of the ATP receptor P2×4, and the chemokine fractalkine, released from neuronal membranes to act upon the fractalkine receptor CX3CR1 found on microglia. Inoue, Glia 40: 156-163, 2002; Tsuda et al., Glia 45: 89-95, 2004; Chapman et al., J Neurosci 20: RC87, 2000; Harrison et al., Proc Natl Acad Sci USA 95: 10896-10901, 1998.

In conclusion, the results show that p38β in the spinal cord play an acute and central role in the cascades initiated by tissue injury and inflammation. The functional association of p38β and its presence in microglia provides strong support for the hypothesis that these cells play a substantial role in spinal nociceptive processing. Development of a centrally active p38β selective inhibitor would contribute to the understanding the function of this isoform and could constitute a potential therapeutic target for pain relief.

### Example 9

### Experimental Procedures

All experiments were carried out according to protocols approved by the Institutional Animal Care Committee of University of California, San Diego.

Animals. Male Holzman Sprague-Dawley rats (300-350 g) were housed individually in micro isolator filter cages and maintained on a 12-h light/dark cycle with free access to food and water. To permit repeated bolus intrathecal drug delivery, chronic lumbar intrathecal injection catheters (single lumen PE-5, 8.5 cm in length) were implanted through a cisternal exposure under isoflurane anesthesia and externalized as described elsewhere. Hayes et al., J Neurosci Methods 126: 165-173, 2003. To permit intrathecal injection and dialysis of the lumbar intrathecal space, rats were prepared with chronic triple lumen loop dialysis catheters advanced 8.5 cm through a cisternal incision to the lumbar enlargement under isoflurane anesthesia and externalized as described elsewhere. Marsala et al., J Neurosci Methods 62: 43-53, 1995. The intrathecal portion of the dialysis probe consists of a tubular 3 cm cellulose dialysis fibers (Filtral AN69HF, Cobe Laboratories) bent double and connected AT its ends to 7 cm of two lumen of the triple lumen catheter, and the third lumen permits the delivery of intrathecal drug without interrupting dialysis. Studies involving rats with chronic intrathecal dialysis catheters were undertaken 4-5 days after surgery and with single lumen injection catheter at 5-8 days after implantation. For bolus intrathecal injection, all agents were prepared to be delivered in 10 μl followed by 10 μl saline to flush the catheter. Rats were monitored daily and removed from the study if any neurological dysfunction was noted, if there was greater than 10% weight loss over 5 days or if the catheter was occluded. Less than 5% of the animals prepared were so excluded.

Drugs: Preparation and administration. For bolus intrathecal injection, all agents were prepared to be delivered in 10 μl followed by 10 μl saline to flush the catheter. Substance P, 30 nmol, (Sigma, St. Louis, Mo.) was dissolved in physiological saline and SB-203580, 3-30 μg, (EMD Biosciences Inc. La Jolla, Calif.) was dissolved in 5% DMSO (Sigma) and 5% Cremophor EL (Sigma) in saline. Intrathecal injection of vehicles (5% DMSO and 5% Cremophor EL in physiological saline or saline alone) had no effect on nociceptive thresholds/behavior or protein expression/phosphorylation.

Antisense oligonucleotides. Three oligonucleotides (a kind gift from ISIS Pharmaceuticals Inc.) with lengths of 20 nucleotides were employed in the study: ISIS 101757 (p38α AS) AGGTGCTCAGGACTCCATTT, beginning at position 1081 in the rat p38α mRNA; ISIS 107211 (p38βAS) GTATGTCCTCCTCGCGTGGA, beginning at position 439 in the rat p38β mRNA; and ISIS141923 (an universe MS control) CCTTCCCTGAAGGTTCCTCC. These oligonucleotides were synthesized as 2′-methoxyethyl (MOE) phosphorothioate chimeric oligonucleotides in which the first and last five bases contained MOE modifications with a uniform phosphorothioate backbone. Butler et al., Diabetes 51: 1028-1034, 2002. The oligonucleotides were dissolved in artificial cerebrospinal fluid (ACSF) immediately prior to intrathecal administration. The ACSF contained (mM) 151.1 Na+, 2.6 K+, 0.9 Mg2+, 1.3 Ca 2+, 122.7 Cl−, 21.0 HCO3, 2.5 HPO₄ and was bubbled with 95% O2/5% CO₂ before use to adjust the final pH to 7.2. AS and MS (10-30 μg) was administrated in 10 μl ACSF followed by 10 μl saline daily for five days and behavioral experiments and spinal tissue collection performed on day six.

Western blot. Prior to sacrifice, rats were deeply anesthetized and after decapitation the spinal cords were ejected from the vertebral column by a saline-filled syringe. The lumbar part of the spinal cord was immediately homogenized in extraction buffer (50 mM Tris buffer, pH 8.0, containing 0.5% Triton X-100, 150 mM NaCl, 1 mM EDTA, phosphatase and protease inhibitors) by sonication. The tissue extracts were subjected to denaturing NuPAGE 4-12% Bis-Tris gel electrophoresis and then transferred to nitrocellulose membranes (Micronic Separation Inc. Westborough, Mass.). After blocking nonspecific binding sites with 5% low-fat milk in PBS containing 0.1% Tween 20 (PBS-T) for 1 hour in room temperature, the membranes were incubated with antibodies overnight at 4° C. After washing, the antibody-protein complexes were probed with appropriate secondary antibodies labeled with horseradish peroxidase for 1 hr at room temperature and detected with chemiluminescent reagents (SuperSignal, Pierce, Rockford, Ill.). The nitrocellulose membranes were stripped with Re-Blot Western blot recycling kit (Chemicon, Temecula, Calif.) and reblotted with different antibodies; phosphorylated p38 (1:1000), p38α (1:500), p38β (1:1000) (Zymed, San Francisco, Calif.), total p38 (1:1000) (Cell Signaling Technology, Beverly, Mass.) and β-actin (1:5000) (Sigma). The p38β antibody is currently characterized only for mouse protein. To confirm that the p38β antibody recognizes rat p38β, spinal cord samples from adult Holzman Sprague-Dawley rats and C3H/HeJ mice (Jackson Laboratory, Bar Harbor, Me.) were subjected to western blotting. Intensity of immunoreactive bands was quantified using ImageQuant software (Molecular Dynamics, Sunnyvale, Calif.). p38 immuno-positive bands were normalized relative to β-actin.

Immunohistochemistry. Animals were deeply anesthetized with sodium pentobarbital (50 mg/kg body weight) and perfused intracardially with heparinized saline (200 ml) followed by freshly prepared 4% paraformaldehyde in 0.1 M phosphate-buffered saline (PBS; pH 7.4; Sigma). The lumbar spinal cord was removed, postfixed in the same fixative for 6 h and transferred to PBS containing 20% sucrose for 24 hours and then 30% sucrose for 48 hours. The lumbar segments L3-6 were dissected, and transverse sections (10 μm) were cut with a freezing microtome and mounted on silane covered glass slides. Non-specific binding was blocked incubation in 5% normal goat serum in PBS-T (PBS with 0.2% Triton X-100) followed by incubation with primary p38α antibody (generated in rabbit, 1:500, Cell Signaling Technology, Beverly, Mass.) overnight in 4° C. under gentle agitation. Binding sites were visualized with anti-rabbit IgG antibodies conjugated with Alexa-488 (1:250). To determine the cellular distribution of p38α, neurons, astrocytes and microglia were counterstained with primary antibodies raised in mouse against markers for neurons: neuronal N (NeuN, 1:1000, Chemicon, Temecula, Calif.), astrocytes: glial fibrillary acid protein (GFAP, 1:500, Chemicon, Temecula, Calif.) and microglia CD11b (OX-42, 1:100 Biosource International), respectively. Binding sites were visualized with anti-mouse IgG antibody conjugated Alexa-594 (1:250). To determine the cellular distribution of p38β, antigen retrieval was performed by microwaving slides for 30 sec while covered with sodium citrate buffer (pH 6), followed by incubation with p38β antibody (1:500, Zymed, San Francisco, Calif.). Binding sites were visualized with anti-mouse IgG antibodies conjugated with Alexa-488. These sections were counterstained with primary antibodies raised in mouse against neuronal marker (NeuN, 1:1000, Chemicon, conjugated to biotin) detected with avidin conjugated to Cy3 (1:1000, Sigma) and astrocyte marker (GFAP, 1:1000, Sigma, labeled with Cy3). Microglia was detected using primary antibody raised in rabbit against a microglia specific calcium binding protein (Iba-1, 1:1000, Wako Chemicals, Richmond, Va.) and binding sites visualized using rabbit IgG antibody conjugated to Alexa-594 (1:250). All antibodies were diluted in 0.5% Triton X-100, 5% and goat serum in PBS. Reagents conjugated to Alexa fluorophores were purchased from Molecular probe, Eugene, Oreg. Cover slips were mounted on the glass slides with ProLong antifade medium (Molecular Probes). Non-specific staining was determined by excluding the primary antibodies. Images were captured using a confocal microscopy system (Nikon) operated by Biorad Lasersharp 2000 software

Nociceptive models. To directly induce spinal sensitization, rats received intrathecal injections of sP (30 nmol) which activates spinal NK-1 receptors and produces thermal hyperalgesia. Hua et al., Neuroscience 89: 525-534, 1999a; Piercey et al., Science 214: 1361-1363, 1981; Yashpal and Henry, Can J Physiol Pharmacol 61: 303-307, 1983. Thermally evoked paw withdrawal responses were assessed using a Hargreaves type testing device. Dirig et al., J Neurosci Methods 76: 183-191, 1997. Briefly, rats were placed on a glass surface maintained at 30° C. The thermal nociceptive stimulus originates from a projection bulb below the glass surface, and the stimulus is delivered separately to one hind paw at a time. The animals were allowed to acclimatize on the glass surface and basal paw withdrawal latencies (PWL) were then assessed for left and right paws at time (T)=−40, =30 and −20 min and expressed as the mean of the six measurements. At T=−10 min the animals received intrathecal vehicle or drug and at T=0, sP was injected. Withdrawal latencies were then assessed at T=15, 30, 45 and 60 min. Paw withdrawal latency for each group was expressed as the mean at each time point.

To quantify formalin induced flinching, an automated sensing system was employed. Yaksh et al., J Appl Physiol 90: 2386-2402, 2001b. Briefly, a soft metal band was placed on the hind paw of the animal being tested. Animals were allowed to acclimate in individual Plexiglas chambers for 30 min before being moved to a test chamber. Just before the animals were placed into the test chamber, they were briefly restrained in a cloth towel, and 2.5% formalin (50 μl) was injected into the dorsal side of the banded paw. Nociceptive behavior was quantified by automatically counting the incidences of spontaneous flinching or shaking of the injected paw. The flinches were counted for 1-min periods for 60 minutes and the flinch data are expressed as number of flinches per min and total flinches observed during phase I (0-9 min) and phase II (10-60 min).

Intrathecal Dialysis & PGE₂ assay. Dialysis experiments were conducted in unanethetized rats 4-5 days after the implant. A syringe pump (Harvard, Natick, Mass.) was connected and dialysis tubing was perfused with artificial cerebrospinal fluid (ACSF) at a rate of 10 μl/min. The ACSF contained (mM) 151.1 Na⁺, 2.6 K⁺, 0.9 Mg²⁺, 1.3 Ca²⁺, 122.7 Cl⁻, 21.0 HCO₃, 2.5 HPO₄ and 3.5 dextrose, and it was bubbled with 95% O2/5% CO₂ before each experiment to adjust the final pH to 7.2. The efflux (15 min per fraction) was collected in an automatic fraction collector (Eicom, Kyoto, Japan) at 4° C. Two baseline samples were collected following a 30-min washout, and additional four fractions after IT injection of SP (30 nmol in 10 III saline followed by 10 μl of saline to flush injection line). The concentration of PGE₂ in spinal dialysate was measured by ELISA using a commercially available kit (Assay Designs 90001, Assay Designs, Ann Arbor, Mich.). The antibody is selective for PGE₂ with less than 2.0% cross-reactivity to PGF_{1α}, PGF_{2α}, 6-ketoPGF_{1α}PGA₂ or PGB₂, but cross-reacts with PGE₁ and PGE₃.

Statistics. For measurements of protein levels by Western blotting five to six animals were included per group and there were four to eight animals in each group for analysis of behavior and PGE₂ release. Differences between groups were compared with one-way ANOVA and Turkey post hoc test except for IT SP-evoked PGE2 release where a unpaired student's t-test was applied (Prism statistical software) and IT SP-evoked thermal hyperalgesia where a two-way ANOVA repeated measurements with a Bonferroni post hoc test were used (StatView statistical software).

When ranges are used herein for physical properties, such as molecular weight, or chemical properties, such as chemical formulae, all combinations and subcombinations of ranges and specific embodiments therein are intended to be included.

The disclosures of each patent, patent application and publication cited or described in this document are hereby incorporated herein by reference in their entirety.

Those skilled in the art will appreciate that numerous changes and modifications can be made to the embodiments of the invention and that such changes and modifications can be made without departing from the spirit of the invention. It is, therefore, intended that the appended claims cover all such equivalent variations as fall within the true spirit and scope of the invention.

What is claimed:

1. A method for preventing or treating pain in a mammal comprising administering an inhibitor of p38β MAP kinase in a therapeutically effective amount to the mammal in need thereof.

2. The method of claim 1, wherein the inhibitor is interfering RNA, short hairpin RNA, ribozyme, antisense oligonucleotide, or protein inhibitor.

3. The method of claim 1, wherein the inhibitor is a monoclonal antibody, a polyclonal antibody, a peptide, or a small molecule.

4. The method of claim 1 wherein the inhibitor is a compound that decreases the enzymatic activity or phosphorylation level of a p38β MAP kinase in the central nervous system of the mammal.

5. The method of claim 1 wherein the pain is hyperalgesia, allodynia or a nociceptive event.

6. The method of claim 1 wherein the inhibitor exhibits an IC₅₀ value for p38β kinase that is at least ten fold less than the IC₅₀ value the inhibitor exhibits relative to other isoforms of p38 MAP kinase.

7. The method of claim 1, further comprising administering an inhibitor of p38α c MAP kinase.

8. The method of claim 4, wherein the compound is administered intrathecally, intramedullarly, intracerebrally, intracerebroventricularly, intracranially, epidurally, intraspinally, or intraparietally.

9. The method of claim 8, wherein the compound crosses the blood-brain barrier of the mammal.

10. The method of claim 4, wherein the compound is administered systemically.

11. The method of claim 10, wherein said contacting comprises administering the compound intravenously, parenterally, subcutaneously, intramuscularly, ophthalmicly, intraventricularly, intraperitoneally, orally, topically, or intranasally to the mammal.

12. The method of claim 1, wherein the p38β MAP kinase inhibitor is administered in an encapsulated form in a lipophilic compound or liposome.

13. The method of claim 1, wherein the MAP kinase inhibitor is encapsulated in a polymer.

14. A method for preventing a facilitative state for sensation of pain in a mammal comprising administering an inhibitor of p38β MAP kinase in a therapeutically effective amount to the mammal in need thereof.

15. The method of claim 14, wherein the inhibitor is interfering RNA, short hairpin RNA, ribozyme, antisense oligonucleotide, or protein inhibitor.

16. The method of claim 14, wherein the inhibitor is a monoclonal antibody, a polyclonal antibody, a peptide, or a small molecule.

17. The method of claim 14 wherein the inhibitor exhibits an IC₅₀ value for p38β kinase that is at least ten fold less than the IC₅₀ value the inhibitor exhibits relative to other isoforms of p38 MAP kinase.

18. The method of claim 14, further comprising administering an inhibitor of p38α MAP kinase.

19. A method of treating, reducing, or preventing pain in a mammalian subject comprising contacting the periphery of the mammalian subject with a compound that decreases the enzymatic activity or phosphorylation level of a p38β MAP kinase in the central nervous system of the mammal, in an amount sufficient to treat, reduce, or prevent pain.

20. The method of claim 19, wherein the compound is administered intrathecally, intramedullarly, intracerebrally, intracerebroventricularly, intracranially, epidurally, intraspinally, or intraparietally.

21. The method of claim 19, wherein the compound crosses the blood-brain barrier of the mammal.

22. The method of claim 19, wherein the compound is administered systemically.

23. The method of claim 19, wherein said contacting comprises administering the compound intravenously, parenterally, subcutaneously, intramuscularly, ophthalmicly, intraventricularly, intraperitoneally, orally, topically, or intranasally to the mammal.

24. The method of claim 19, further comprising administering an inhibitor of p38α MAP kinase.

25. A method for identifying a compound which inhibits p38β MAP kinase comprising:
contacting a test compound with a cell-based assay system comprising a cell expressing p38β MAP kinase and capable of signaling responsiveness to p38β MAP kinase
detecting an effect of the test compound on p38β MAP kinase signaling in the assay system, effectiveness of the test compound in the assay being indicative of the inhibition.

26. The method of claim 25, wherein the test compound is a monoclonal antibody, a polyclonal antibody, a peptide, a nucleic acid, or a small molecule.

27. A pharmaceutical composition comprising a p38β MAP kinase inhibitor for treatment of pain.

28. The composition of claim 27 wherein the pain is hyperalgesia.

29. The composition of claim 27 wherein the pain is allodynia.

30. The composition of claim 27 wherein the inhibitor is interfering RNA, short hairpin RNA, ribozyme, antisense oligonucleotide, or protein inhibitor.

31. The composition of claim 27 wherein the inhibitor is a monoclonal antibody, a polyclonal antibody, a peptide, or a small molecule.

32. The composition of claim 30 wherein the antisense oligonucleotide is 5′-GTATGTCCTCCTCGCGTGGA-3′.
